(19) United States
(12) Patent Application Publication
(10) Pub. No.: US 2018/0130957 A1
(43) Pub. Date: May 10, 2018

(54) Organic Electroluminescence Device

(71) Applicant: UDC IRELAND LIMITED, Dublin (IE)

(72) Inventors: Toru WATANABE, Kanagawa-ken (JP); Tetsu KITAMURA, Kanagawa-ken (JP); Toshihiro ISE, Kanagawa-ken (JP)

(21) Appl. No.: 15/859,970

(22) Filed: Jan. 2, 2018

Related U.S. Application Data
(63) Continuation of application No. 13/522,056, filed on Jul. 13, 2012.

(30) Foreign Application Priority Data
Jan. 15, 2010 (JP) 2010-007534
May 20, 2010 (JP) 2010-116664

(51) Int. Cl. H01L 51/00; C07D 403/10; C07F 15/00; C09K 11/06; H05B 33/14; C07D 209/82; H01L 51/50

(52) U.S. Cl. CPC H01L 51/0072; C07D 403/10; C07F 15/0033; C09K 11/06; H05B 33/14; H01L 51/0085; C09K 2211/1029; C09K 2211/1007; C09K 2211/1011; C09K 2211/185; H01L 51/0054; H01L 51/0081; H01L 51/5016; C07D 209/82

(57) ABSTRACT

As an organic electroluminescence device that exhibits superior external quantum efficiency and durability during driving at high temperature, and small variation in chromaticity and small increase in voltage after driving at high temperatures and has long lifespan, it is provided that the organic electroluminescence device including on a substrate a pair of electrodes and at least one layer of an organic layer including a light emitting layer disposed between the electrodes, wherein the light emitting layer contains at least one specific iridium complex and any layer of the at least one layer of an organic layer contains at least one compound represented by Formula (1):

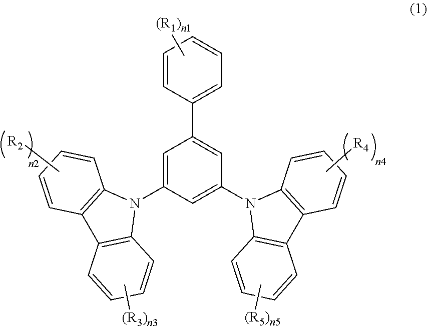

wherein in Formula (1), each of R₁ to R₅ independently represents a specific group or atom, n1 represents an integer of 0 to 5, and each of n2 to n5 independently represents an integer of 0 to 4.

### TECHNICAL FIELD

The present invention relates to an organic electroluminescence device (hereinafter, also referred to as a “device” or an “organic EL device”). More specifically, the present invention relates to an organic electroluminescence device with long lifespan that exhibits superior properties of devices required for driving at high temperatures (specifically, external quantum efficiency, durability, variation in chromaticity and variation in voltage) and excellent long lifespan.

### BACKGROUND ART

An organic electroluminescence device is being actively researched and developed, since it can emit light with high luminance intensity through driving at a low voltage. Generally, the organic electroluminescence device includes an organic layer including a light emitting layer and a pair of electrodes disposed via the layer and emits light using energy of excitons produced by recombination of electrons injected from a cathode with holes injected from an anode in the light emitting layer.

Recently, efficiency of device is increased using a phosphorescent light emitting material. For example, organic electroluminescence devices that exhibit improved light emitting efficiency and heat resistance through use of an iridium or platinum complex as the phosphorescent light emitting material is researched.

In addition, a dope-type device using a light emitting layer in which a light emitting material is doped into a host material is widely used.

Recently, host materials are actively developed and, for example, Patent Document 1 discloses a device using a cabazole compound in which a plurality of aryl groups are combined to one another, as a host material, for the purpose of manufacturing devices that have superior light emitting efficiency, decreased pixel defects and excellent heat resistance.

In addition, Patent Document 2 discloses an invention using an aromatic polycyclic condensed ring-based material as a host material and using a phenylquinoline-based red phosphorescent material as a dopant, for manufacturing devices with high efficiency and long lifespan. Patent Document 2 discloses an invention using an electron transporting material having a carbazole structure. This document discloses that it is not preferable to incorporate a carbazole group generally vulnerable to oxidation since it leads to shortening of lifespan of devices.

However, conventional devices have problems of low durability during driving at high temperatures, and large variation in chromaticity and a large increase in voltage after driving at high temperatures. For this reason, there is a need for solutions to these problems.

### RELATED ART

### Patent Document

Patent Document 1: WO 04/074399

Patent Document 2: JP-A-2009-99783

### DISCLOSURE OF INVENTION

### Problems to be Solved by the Invention

As described in Patent Document 2, use of material having a carbazole group generally vulnerable to oxidation is known to be not preferred in terms of lifespan of devices. In the embodiment of the present invention, it is thought that problems associated with the lifespan of device occur in consideration of the common knowledge.

In addition, conventional devices should be improved due to problems of low durability during driving at high temperature, and large variation in chromaticity and large increase in voltage after driving at high temperatures.

The inventors of the present invention discovered that devices with a long lifespan are unexpectedly obtained when a host material containing a carbazole group of the present invention is used in combination with a specific iridium complex material.

In addition, the inventors of the present invention discovered that the configuration of the present invention provides a device that exhibits high external quantum efficiency and durability during driving at high temperature, and decreased variation in chromaticity and small increase in voltage after driving at high temperatures.

That is, it is one object of the present invention to provide an organic electroluminescence device that exhibits superior external quantum efficiency and durability during driving at high temperature and decreased variation in chromaticity and small increase in voltage after driving at high temperatures and has long lifespan.

In addition, it is another object of the present invention to provide a light emitting layer and a composition useful for the organic electroluminescence device. Furthermore, it is another object of the present invention to provide a light emission apparatus, display apparatus and an illumination apparatus including the organic electroluminescence device.

### Means for Solving the Problems

That is, the present invention is accomplished by the following means.

[1] An organic electroluminescence device, comprising on a substrate:

a pair of electrodes; and

at least one layer of an organic layer including a light emitting layer disposed between the electrodes,

wherein the light emitting layer contains at least one compound represented by Formula (PQ-1), and

any layer of the at least one layer of an organic layer contains at least one compound represented by Formula (1):

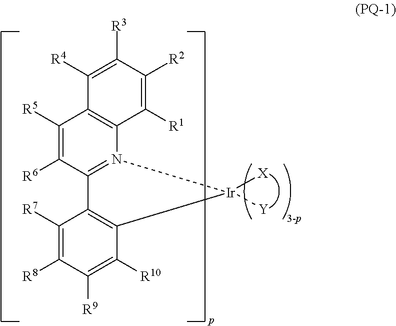

wherein in Formula (PQ-1), each of R¹ to R¹⁰ independently represents a hydrogen atom, an alkyl group, a cycloalkyl group, an aryl group, a cyano group or a fluorine atom, and substituents represented by R¹ to R¹⁰ may be combined together to form a ring, provided that all of the substituents represented by R¹ to R¹⁰ are not a hydrogen atom at the same time;

(X-Y) represents a monoanionic bidentate ligand; and

p represents an integer of 1 to 3:

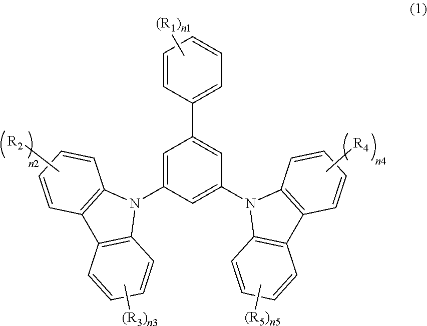

wherein in Formula (1), R₁ represents an alkyl group, an aryl group or a silyl group and may further have a substituent Z, provided that R₁ does not represent a carbazolyl group or a perfluoroalkyl alkyl group, and when R₁ is present in plural, each of a plurality of R₁'s may be the same as or different from every other R₁, and a plurality of R₁'s may be combined together to form an aryl ring which may have a substituent Z;

each of R₂ to R₅ independently represents an alkyl group, an aryl group, a silyl group, a cyano group or a fluorine atom and may further have a substituent Z, and when each of R₂ to R₅ is present in plural, each of a plurality of R₂'s to a plurality of R₅'s may be the same as or different from every other R₂ to R₅, respectively;

the substituent Z represents an alkyl group, an alkenyl group, an aryl group, an aromatic heterocyclic group, an alkoxy group, a phenoxy group, a fluorine atom, a silyl group, an amino group, a cyano group or a group formed by a combination thereof, and a plurality of the substituent Z's may be combined together to form an aryl group;

n1 represents an integer of 0 to 5; and

each of n2 to n5 independently represents an integer of 0 to 4.

[2] The organic electroluminescence device as described in [1] above,

wherein, in Formula (PQ-1), p is 2.

[3] The organic electroluminescence device as described in [1] or [2] above,

wherein in Formula (PQ-1), the monoanionic bidentate ligand (X-Y) is represented by the following Formula (PQL-1):

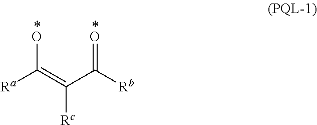

wherein in Formula (PQL-1), each of R^{a} to R^{c} independently represents a hydrogen atom or an alkyl group; and

* represents a position coordinated to iridium.

[4] The organic electroluminescence device as described in any one of [1] to [3] above,

wherein in Formula (PQ-1), R¹ to R⁶ represent a hydrogen atom, each of R⁷ to R¹⁰ independently represents a hydrogen atom, an alkyl group or an aryl group, and at least one of R⁷ to R¹⁰ represents an alkyl group or an aryl group.

[5] The organic electroluminescence device as described in any one of [1] to [4] above,

wherein the compound represented by Formula (1) is used in the light emitting layer.

[6] The organic electroluminescence device as described in any one of [1] to [5],

wherein the compound represented by Formula (1) is represented by the following Formula (2):

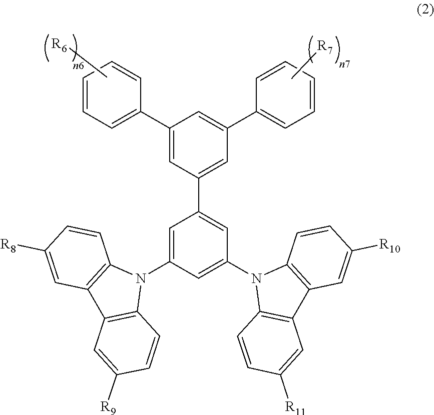

wherein, in Formula (2), each of R₆ and R₇ independently represents an alkyl group which may have a substituent Z, an aryl group which may have an alkyl group, a cyano group or a fluorine atom, and when each of R₆ and R₇ is present in plural, each of a plurality of R₆'s and a plurality of R₇'s may be the same as or different from every other R₆ and R₇, respectively, and each of the plurality of R₆'s and the plurality of R₇'s may be combined together to form an aryl ring that may have a substituent Z;

each of n6 and n7 independently represents an integer of 0 to 5;

each of R₈ to R₁₁ independently represents a hydrogen atom, an alkyl group which may have a substituent Z, an aryl group which may have an alkyl group, a silyl group which may have a substituent Z, a cyano group or a fluorine atom; and

the substituent Z represents an alkyl group, an alkenyl group, an aryl group, an aromatic heterocyclic group, an alkoxy group, a phenoxy group, a fluorine atom, a silyl group, an amino group, a cyano group or a group formed by a combination thereof, and a plurality of substituent Z's may be combined together to form an aryl group.

[7] The organic electroluminescence device as described in [6] above,

wherein in Formula (PQ-1), R¹ to R⁶ represent a hydrogen atom, each of R⁷ to R¹⁰ independently represents a hydrogen atom, an alkyl group or an aryl group, at least one of R⁷ to R¹⁰ represents an alkyl group or an aryl group, p is 2 and the monoanionic bidentate ligand (X-Y) is represented by Formula (PQL-1), and in Formula (PQL-1), each of R^{a} and R^{b} independently represents an alkyl group, R^{c} represents a hydrogen atom, and in Formula (2), each of R₆ and R₇ independently represents an alkyl group or an aryl group which may have an alkyl group, each of n6 and n7 independently represents an integer of 0 to 2, each of R₈ to R₁₁ independently represents a hydrogen atom, an alkyl group, an aryl group which may have an alkyl group, a silyl group substituted by an alkyl group or a phenyl group, a cyano group or a fluorine atom.

[8] The organic electroluminescence device as described in any one of [1] to [7] above, further comprising:

an electron injection layer disposed between the electrodes,

wherein the electron injection layer contains an electron donating dopant.

[9] The organic electroluminescence device as described in any one of [1] to [7] above, further comprising:

a hole injection layer disposed between the electrodes,

wherein the hole injection layer contains a hole accepting dopant.

[10] The organic electroluminescence device as described in any one of [1] to [9] above,

wherein at least one layer of the organic layer disposed between the pair of electrodes is formed by a solution coating process.

[11] A light emitting layer, comprising:

the compound represented by Formula (PQ-1) and the compound represented by Formula (1) or (2) as described in any one of [1] to [10] above.

[12] A composition, comprising:

the compound represented by Formula (PQ-1) and the compound represented by Formula (1) or (2) as described in any one of [1] to [10] above.

[13] A light emission apparatus using the organic electroluminescence device as described in any one of [1] to [10] above.

[14] A display apparatus using the organic electroluminescence device as described in any one of [1] to [10] above.

[15] An illumination apparatus using the organic electroluminescence device as described in any one of [1] to [10] above.

### Effects of the Invention

The organic electroluminescence device of the present invention exhibits superior device properties during driving at high temperatures. Specifically, the organic electroluminescence device of the present invention exhibits superior external quantum efficiency and high durability during driving at high temperatures, and small variation in chromaticity and small increase in voltage after driving at high temperatures. In addition, according to the present invention, the organic electroluminescence device with a long lifespan can be provided even when materials having a carbazole group are used as host materials.

### BRIEF DESCRIPTION OF DRAWINGS

FIG. 1 is a schematic view illustrating one example of a layer structure of an organic EL device according to the present invention (a first embodiment);

FIG. 2 is a schematic view illustrating one example of a light emission apparatus according to the present invention (a second embodiment); and

FIG. 3 is a schematic view illustrating one example of an illumination apparatus according to the present invention (a third embodiment).

### EMBODIMENTS FOR CARRYING OUT THE INVENTION

Hereinafter, in the description of Formula (PQ-1), Formula (PQL-1), Formula (1) and Formula (2), a hydrogen atom includes an isotope (such as heavy hydrogen atom) and, furthermore, an atom constituting a substituent also includes an isotope thereof.

In the present invention, the substituent Z is defined as below.

### (Substituent Z)

The substituent Z represents an alkyl group, an alkenyl group, an aryl group, aromatic heterocyclic group, an alkoxy group, a phenoxy group, a fluorine atom, a silyl group, an amino group, a cyano group or a group formed by combination thereof. A plurality of substituents Z may be combined together to form an aryl group.

The alkyl group represented by the substituent Z preferably is an alkyl group having 1 to 8 carbon atoms, more preferably, an alkyl group having 1 to 6 carbon atoms. Examples of the alkyl group include a methyl group, an ethyl group, an n-propyl group, an isopropyl group, an isobutyl group, a t-butyl group, an n-butyl group, a cyclopropyl group and the like. A methyl group, an ethyl group, isobutyl group or t-butyl group is preferred and a methyl group is more preferred.

The alkenyl group represented by the substituent Z preferably is an alkenyl group having 2 to 8 carbon atoms, more preferably an alkenyl group having 2 to 6 carbon atoms. Examples of the alkenyl group include a vinyl group, an n-propenyl group, an isopropenyl group, an isobutenyl group, an n-butenyl group and the like. A vinyl group, n-propenyl group, isobutenyl group or n-butenyl group is preferred and a vinyl group is more preferred.

The aryl group represented by the substituent Z is preferably an aryl group having 6 to 18 carbon atoms, more preferably an aryl group having 6 to 12 carbon atoms. Examples of the aryl group include a phenyl group, a biphenyl group, a terphenyl group, a naphthyl group, an anthranyl group, a phenanthryl group, a chrysenyl group and the like. Among them, a phenyl group, a biphenyl group, a terphenyl group or a naphthyl group are preferred, a phenyl group, a biphenyl group or a terphenyl group is more preferred, and a phenyl group is even more preferred.

The aromatic heterocyclic group represented by the substituent Z preferably is an aromatic heterocyclic group having 4 to 12 carbon atoms, and more preferably an aromatic heterocyclic group having 5 to 10 carbon atoms. Examples of the aromatic heterocyclic group include a pyridyl group, a furyl group, a thienyl group and the like. A pyridyl group or a furyl group is preferred, and a pyridyl group is more preferred.

The alkoxy group represented by the substituent Z is preferably an alkoxy group having 1 to 8 carbon atoms, more preferably an alkoxy group having 1 to 4 carbon atoms. Examples of the alkoxy group include a methoxy group, an ethoxy group, an n-propoxy group, an isopropoxy group, an isobutoxy group, a t-butoxy group, an n-butoxy group, a cyclopropyloxy group and the like. A methoxy group, an ethoxy group, an isobutoxy group, or a t-butoxy group is preferred and a methoxy group is more preferred.

The silyl group represented by the substituent Z is preferably a silyl group having 3 to 40 carbon atoms, more preferably a silyl group having 3 to 30 carbon atoms, particularly preferably a silyl group having 3 to 24 carbon atoms and examples thereof include a trimethylsilyl group, a triphenylsilyl group and the like.

The amino group represented by the substituent Z is preferably an amino group having 0 to 30 carbon atoms, more preferably an amino group having 0 to 20 carbon atoms, particularly preferably an amino group having 0 to 10 carbon atoms, and examples thereof include an amino group, a methylamino group, a dimethylamino group, a dimethylamino group, a dibenzylamino group, a diphenylamino group, a di tolylamino group and the like.

Examples of the aryl ring formed by combining a plurality of substituents Z together include a benzene ring, a naphthalene ring and the like. A benzene ring is preferred.

The organic electroluminescence device of the present invention is an organic electroluminescence device that includes on a substrate a pair of electrodes; and at least one organic layer including a light emitting layer disposed between the electrodes, wherein the light emitting layer contains at least one compound represented by Formula (PQ-1) and any layer of the at least one organic layer contains at least one compound represented by Formula (1).

[Compound Represented by Formula (PQ-1)]

Hereinafter, the compound represented by Formula (PQ-1) will be described.

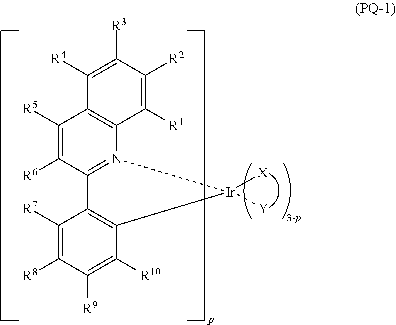

In Formula (PQ-1), each of R¹ to R¹⁰ independently represents a hydrogen atom, an alkyl group, a cycloalkyl group, an aryl group, a cyano group or a fluorine atom. The substituents represented by R¹ to R¹⁰ may be combined together to form a ring. It is provided that all of the substituents represented by R¹ to R¹⁰ are not a hydrogen atom at the same time.

(X-Y) represents a monoanionic bidentate ligand.

p represents an integer of 1 to 3.

Each of the substituents represented by R¹ to R¹⁰ preferably independently represents a hydrogen atom, an alkyl group, a cycloalkyl group, an aryl group or a fluorine atom, more preferably a hydrogen atom, an alkyl group or an aryl group, even more preferably a hydrogen atom or an alkyl group. However, all of the substituents represented by R¹ to R¹⁰ are not a hydrogen atom at the same time.

Each of alkyl groups represented by R¹ to R¹⁰ may independently have a substituent and may be saturated or unsaturated. The substituent of the alkyl group having a substituent may be the aforementioned substituent Z and the substituent Z is preferably a fluorine atom. The alkyl group represented by R¹ to R¹⁰ is preferably an alkyl group having 1 to 8 carbon atoms, more preferably an alkyl group having 1 to 6 carbon atoms, and examples thereof include a methyl group, a trifluoromethyl group, an ethyl group, a vinyl group, an n-propyl group, an isopropyl group, an isobutyl group, a t-butyl group, an n-butyl group and the like. A methyl group, a trifluoromethyl group, an ethyl group, an isopropyl group or t-butyl group is preferred, a methyl group or an ethyl group is more preferred and a methyl group is even more preferred.

Each of the cycloalkyl group represented by R¹ to R¹⁰ may independently have a substituent, and may be saturated or unsaturated. The substituent of the cycloalkyl group having a substituent may be the aforementioned substituent Z and the substituent Z is preferably an alkyl group. The cycloalkyl group represented by R¹ to R¹⁰ is preferably a cycloalkyl group having 3 to 20 carbon atoms, more preferably a cycloalkyl group having 3 to 10 carbon atoms, even more preferably a cycloalkyl group a cycloalkyl group having 5 to 10 carbon atoms. Examples of the cycloalkyl group include a cyclopentyl group, a cyclohexyl group, a cycloheptyl group, a cyclooctyl group, a cyclohexenyl group and the like. A cyclohexyl group or cyclohexenyl group is preferred.

Each of the aryl group represented by R¹ to R¹⁰ may be independently condensed and may have a substituent. The substituent of the aryl group having a substituent may be the aforementioned substituent Z, and the substituent Z is preferably an alkyl group, an aryl group or a fluorine atom, more preferably an alkyl group or an aryl group, even more preferably an alkyl group. The aryl group represented by R¹ to R¹⁰ is preferably an aryl group having 6 to 12 carbon atoms, more preferably an aryl group having 6 to 10 carbon atoms. Examples thereof include a phenyl group, a dimethylphenyl group and the like, and a phenyl group is preferred.

The substituents represented by R¹ to R¹⁰ may be combined together to form a ring. When the ring is formed, the formation is preferably carried out by combining adjacent two groups of R¹ to R¹⁰ together, and is more preferably by combining R⁷ with R⁸ together, or combining R⁸ with R⁹ together. The formed ring is a cycloalkyl ring, an aryl ring or the like, which may have the aforementioned substituent Z and may be further condensed with an aryl group. The substituent Z is preferably an alkyl group, an aryl group or a fluorine atom.

The formed cycloalkyl ring, including carbon atoms associated with the formation of ring other than R¹ to R¹⁰, is preferably a cycloalkyl ring having 5 to 30 carbon atoms, more preferably a cycloalkyl ring having 5 to 14 carbon atoms. Examples of the formed cycloalkyl ring include a cyclopentyl ring, a cyclohexyl ring and an indane ring. A cyclohexyl ring or indane ring is preferred and an indane ring is more preferred.

The formed aryl ring, including carbon atoms associated with the formation of ring other than R¹ to R¹⁰, is preferably an aryl ring having 6 to 30 carbon atoms, more preferably an aryl ring having 6 to 14 carbon atoms. Examples of the formed aryl ring include a benzene ring, a naphthalene ring, a phenanthrene ring and the like. A benzene ring or phenanthrene ring is preferred and a benzene ring is more preferred.

In Formula (PQ-1), preferably, each of zero to three of R¹ to R⁶ independently represents an alkyl group, a cycloalkyl group, an aryl group, a cyano group or a fluorine atom and all of the remaining of R¹ to R⁶ are a hydrogen atom, and more preferably, zero or one of R¹ to R⁶ represents an alkyl group, a cycloalkyl group, an aryl group, a cyano group or a fluorine atom and all of the remaining of R¹ to R⁶ are a hydrogen atom, and even more preferably, all of R¹ to R⁶ are a hydrogen atom in terms of improvement of durability.

Preferably, each of zero to two of R⁷ to R¹⁰ independently represents an alkyl group, a cycloalkyl group, an aryl group, a cyano group or a fluorine atom and, at the same time, all of the remaining of R⁷ to R¹⁰ are a hydrogen atom. More preferably, each of zero to two of R⁷ to R¹⁰ independently represents an alkyl group, an aryl group, a cyano group or a fluorine atom and all of the remaining of R⁷ to R¹⁰ are a hydrogen atom. However, when all of R⁷ to R¹⁰ are a hydrogen atom, at least one of R¹ to R⁶ represents an alkyl group, a cycloalkyl group, an aryl group, a cyano group or a fluorine atom. Furthermore, R⁷ and R⁸, or R⁸ and R⁹ may be combined together to form the aforementioned ring. In a case in which the ring is formed, the aforementioned aryl ring is more preferably formed, and a benzene ring is even more preferably formed.

In addition, in order to improve durability, in a case in which all of R¹ to R⁶ are a hydrogen atom, preferably, each of R⁷ to R¹⁰ independently represents a hydrogen atom, an alkyl group or an aryl group and at least one of R⁷ to R¹⁰ is an alkyl group or an aryl group, and more preferably, at least one of R⁷ to R¹⁰ is an alkyl group, and most preferably, two of R⁷ to R¹⁰ are an alkyl group. Also, R⁷ and R⁸, or R⁸ and R⁹ may be combined together to form the aforementioned ring. In a case in which the ring is formed, the aforementioned aryl ring is more preferably formed, and a benzene ring is even more preferably formed.

When all of R¹ to R⁶ are hydrogen atoms, examples of the alkyl group represented by at least one of R⁷ to R¹⁰ include a methyl group, a trifluoromethyl group, an ethyl group, an n-propyl group, an iso-propyl group, an iso-butyl group, a t-butyl group, an n-butyl group and the like. A methyl group, a trifluoromethyl group, an ethyl group, an iso-butyl group or a t-butyl group is preferred, and a methyl group is more preferred. Also, the aryl group is preferably a phenyl group.

In terms of durability, when at least one of R⁷ to R¹⁰ is an alkyl group or an aryl group, at least R⁸ is preferably an alkyl group or an aryl group.

p is preferably 2 or 3 and more preferably 2.

(X-Y) represents a monoanionic bidentate ligand. It is thought that these ligands do not directly contribute to photoactivity and can change photoactivity of molecules. The monoanionic bidentate ligand used as a light emitting material may be selected from those known in the art. Non-limited examples of the monoanionic bidentate ligand are described in WO 02/15645 filed by Lamansky et al., which is incorporated herein by reference, 89 to 90 pages. Preferred monoanionic bidentate ligands include acetylacetonate (acac) and picolinate (pic) and derivatives thereof. In the present invention, in terms of stability of complexes and high light emitting quantum efficiency, the monoanionic bidentate ligand is preferably acetylacetonate represented by Formula (PQL-1) and a derivative thereof.

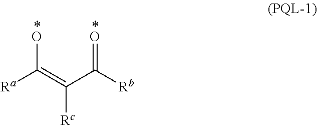

In Formula (PQL-1), each of R^{a} to R^{c} independently represents a hydrogen atom or an alkyl group. * represents a position coordinated to iridium.

The alkyl group represented by R^{a} to R^{c} is preferably an alkyl group having 1 to 8 carbon atoms, more preferably an alkyl group having 1 to 4 carbon atoms. Examples thereof include a methyl group, an ethyl group, an n-propyl group, an isopropyl group, an isobutyl group, a t-butyl group, an n-butyl group, a cyclopropyl group, a trifluoromethyl group and the like. A methyl group, an ethyl group, an isobutyl group or a t-butyl group is preferred, a methyl group or a t-butyl group is more preferred, and a methyl group is even more preferred.

In terms of stability of complexes, each of R^{a} and R^{b} is preferably an alkyl group, more preferably an alkyl group having 1 to 4 carbon atoms, more preferably either a methyl group or a t-butyl group, even more preferably a methyl group. R^{a} and R^{b} are preferably identical.

R^{c} is preferably a hydrogen atom.

Hereinafter, specific examples of the compound represented by Formula (PQ-1) are given below and the present invention is not limited thereto.

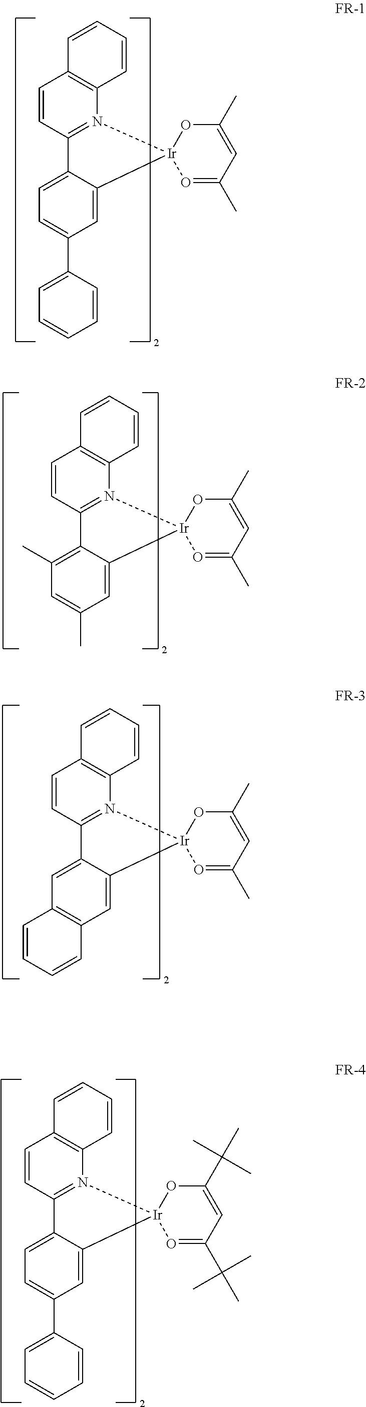

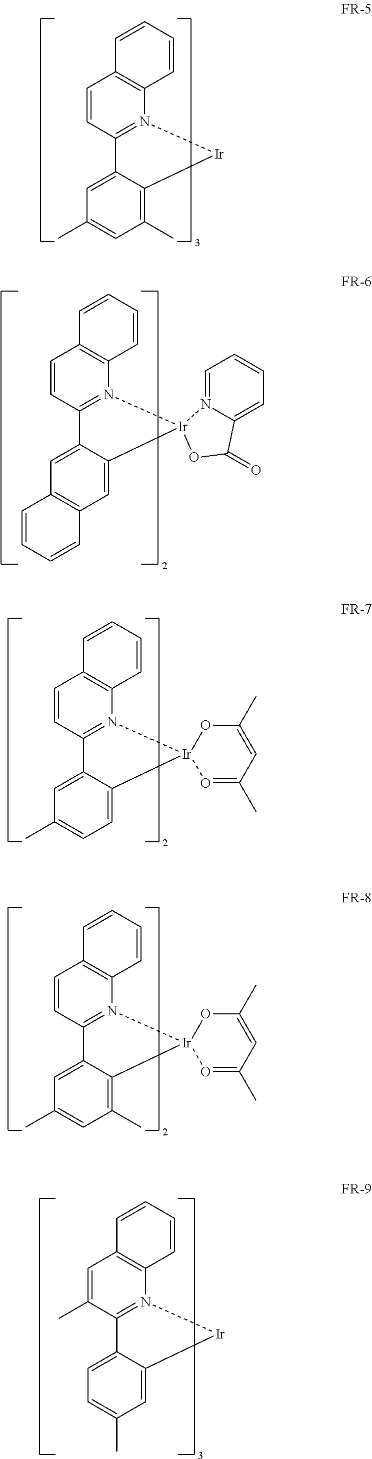

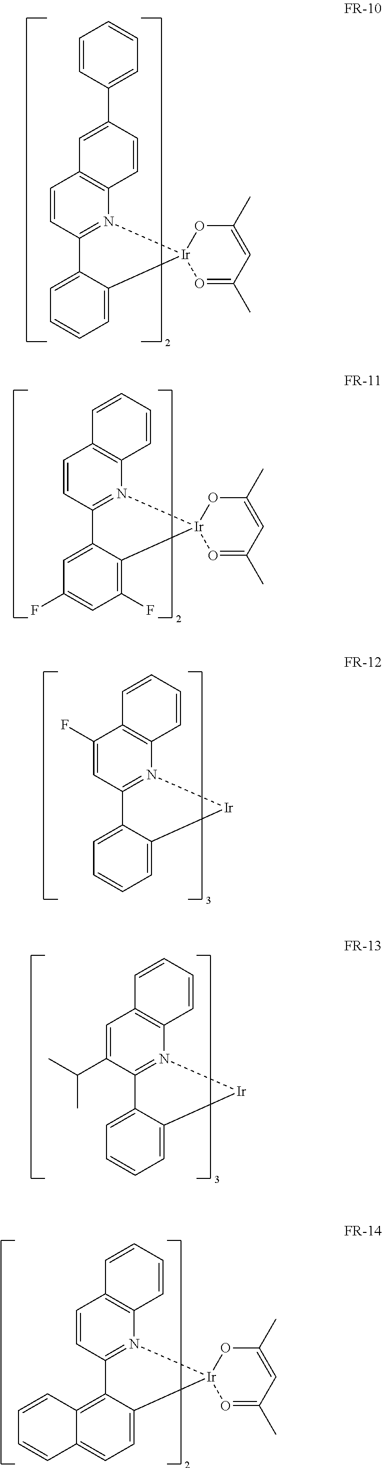

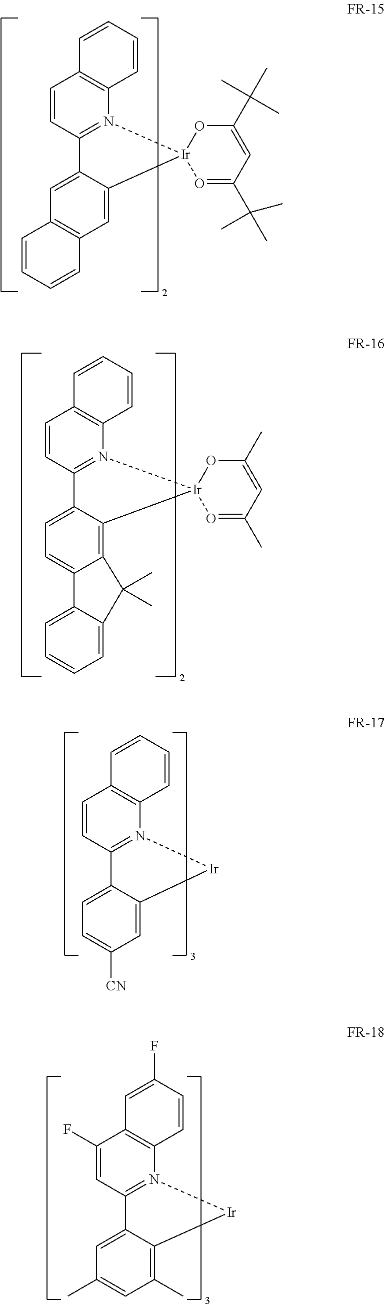

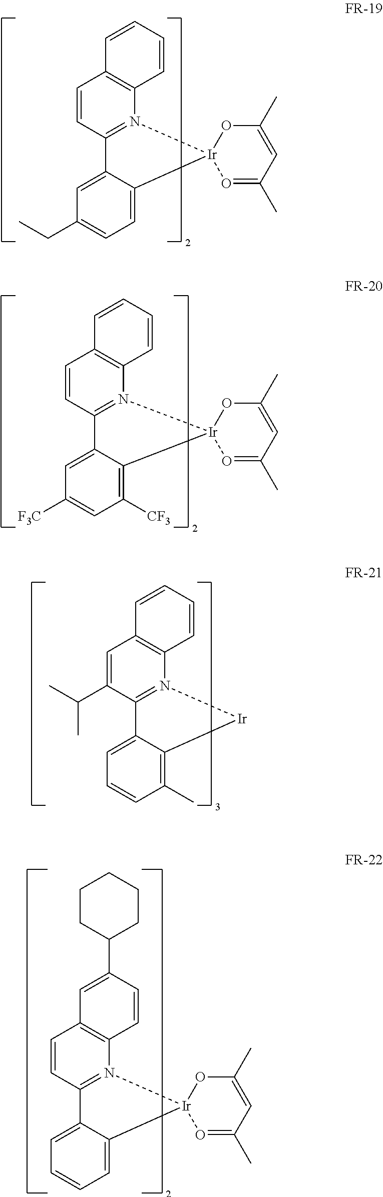

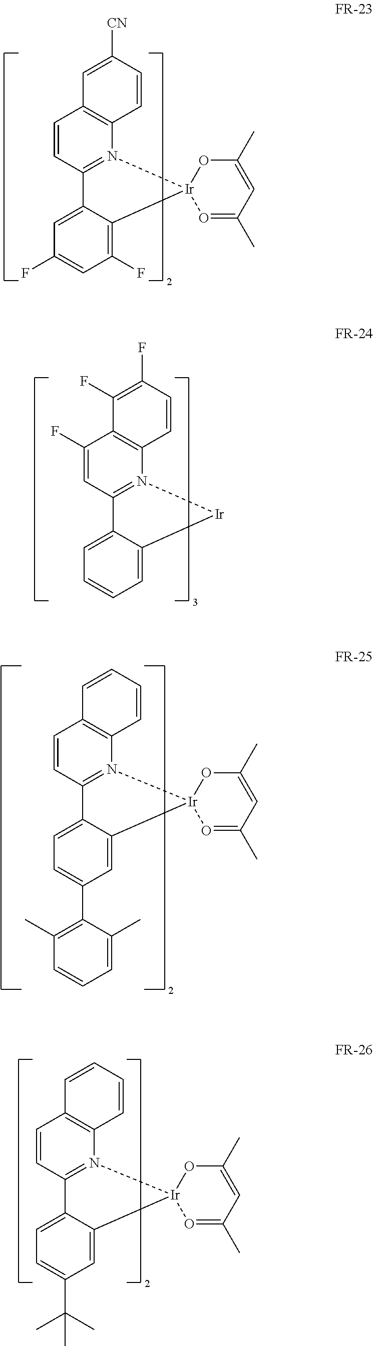

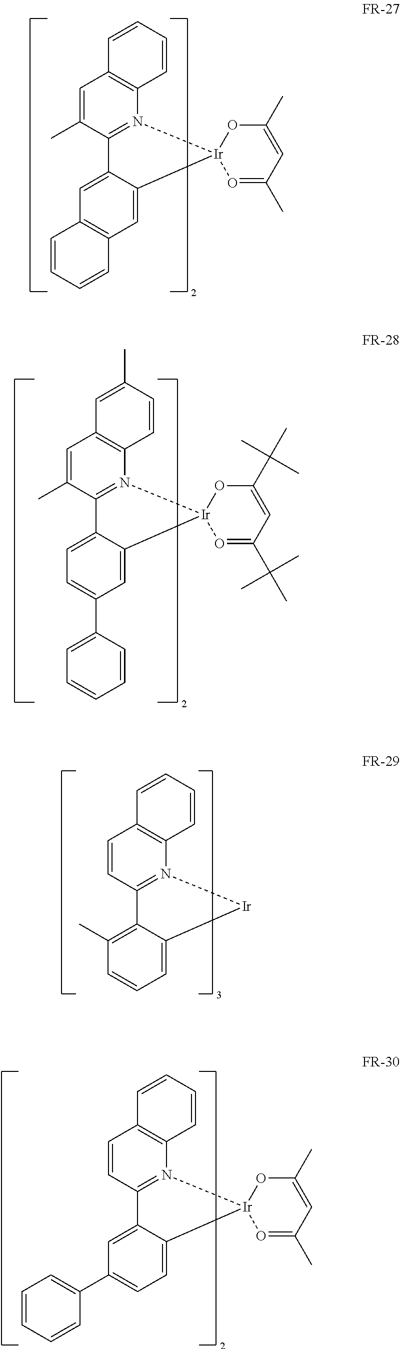

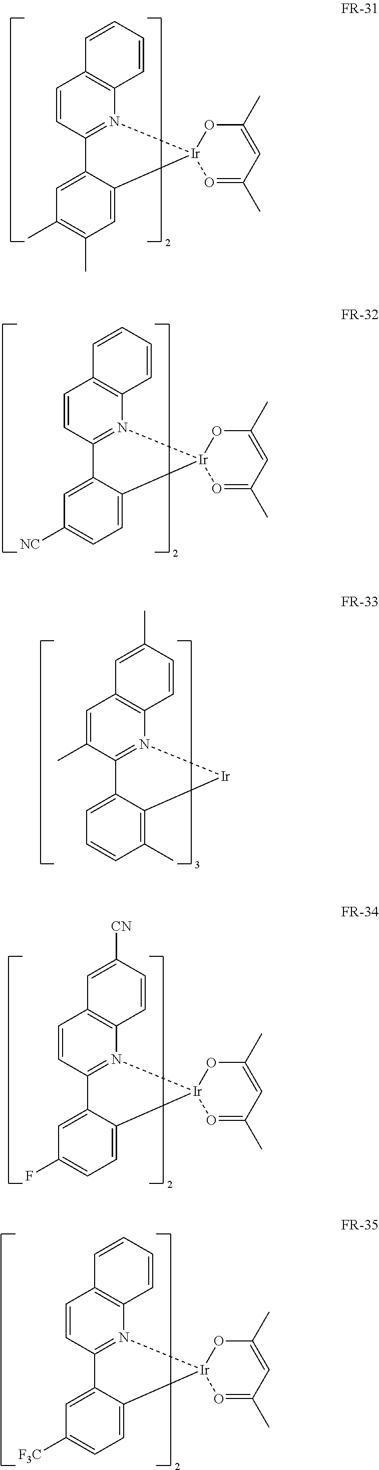

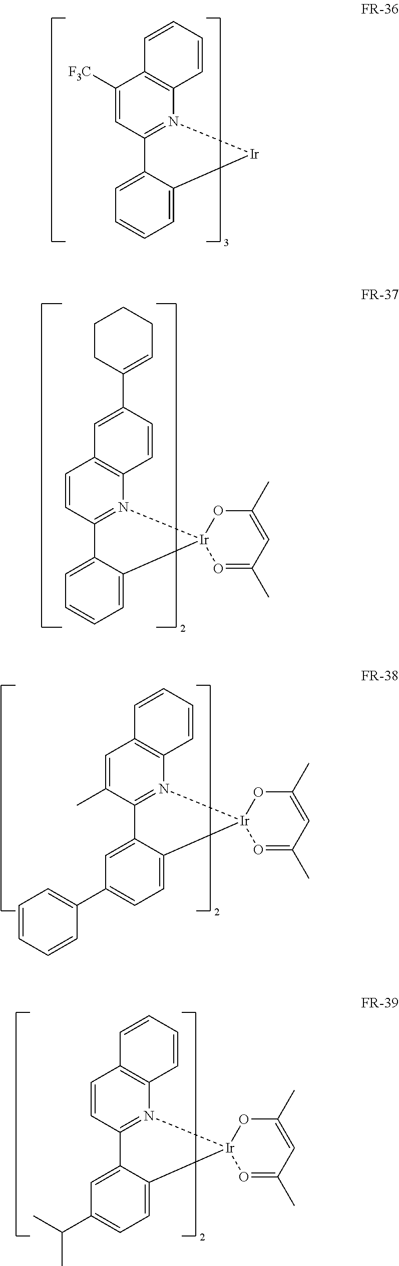

For example, the compounds given as examples of the compound represented by Formula (PQ-1) may be synthesized by the method described in Japanese Patent No. 3929689. For example, FR-2 can be synthesized by the paragraphs [0054] to [0055] (page 18, lines 1 to 13) of Japanese Patent No. 3929689.

In the present invention, the compound represented by Formula (PQ-1) is contained in the light emitting layer and use thereof is not limited and may be further contained in any one of the organic layer.

In the present invention, in order to further suppress variation in chromaticity after driving at high temperatures, a compound represented by Formula (1) or (2) described below and a compound represented by Formula (PQ-1) are preferably contained in the light emitting layer.

The compound represented by Formula (PQ-1) is preferably contained in an amount of 0.1 to 30% by mass, more preferably 1 to 20% by mass, even more preferably 5 to 15% by mass, with respect to the total mass of the light emitting layer.

[Compound Represented by Formula (1)]

Hereinafter, the compound represented by Formula (1) will be described.

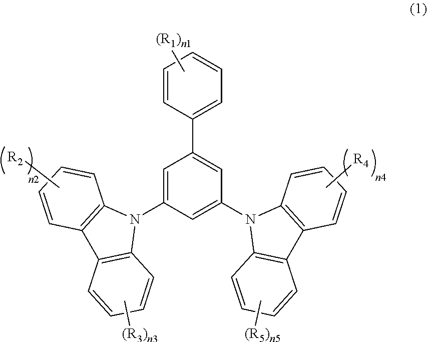

In Formula (1), R₁ represents an alkyl group, an aryl group or a silyl group and may further have a substituent Z. However, there is no case in which R₁ represents a carbazolyl group or a perfluoroalkyl alkyl group. In a case in which R₁ is present in plural, each of a plurality of R₁'s may be the same as or different from every other R₁. In addition, a plurality of R₁'s may be combined together to form an aryl ring which may have a substituent Z.

Each of R₂ to R₅ independently represents an alkyl group, an aryl group, a silyl group, a cyano group or a fluorine atom and may further have a substituent Z. In a case in which each of R₂ to R₅ is present in plural, each of the plurality of R₂'s to the plurality of R₅'s may be the same as or different from every other R₂ to R₅, respectively.

The substituent Z represents an alkyl group, an alkenyl group, an aryl group, an aromatic heterocyclic group, an alkoxy group, a phenoxy group, a fluorine atom, a silyl group, an amino group, a cyano group or a group formed by combination thereof, and the plurality of substituents Z may be combined together to form an aryl group.

n1 represents an integer of 0 to 5.

Each of n2 to n5 independently represents an integer of 0 to 4.

The alkyl group represented by R₁ may have a substituent and may be saturated or unsaturated. The substituent of the alkyl group having a substituent may be the aforementioned substituent Z, and the substituent Z is preferably a fluorine atom. However, there is no case in which the alkyl group represented by R₁ is a perfluoroalkyl alkyl group. The alkyl group represented by R₁ is preferably an alkyl group having 1 to 8 carbon atoms, more preferably an alkyl group having 1 to 6 carbon atoms, even more preferably an alkyl group having 1 to 4 carbon atoms. Examples thereof include a methyl group, an ethyl group, an n-propyl group, an isopropyl group, an n-butyl group, an isobutyl group, a sec-butyl group, a t-butyl group, an n-pentyl group, an isopentyl group, a 2-methylpentyl group, a neopentyl group, a n-hexyl group, a 4-methylpentyl group, a 3-methylpentyl group, a 2-methylpentyl group, a 3,3-dimethylbutyl group, a 2,2-dimethylbutyl group, a 1,1-dimethylbutyl group, a 1,2-dimethylbutyl group, a 1,3-dimethylbutyl group, a 2,3-dimethylbutyl group and the like. Among them, a methyl group, an isopropyl group, a t-butyl group or a neopentyl group is preferred, a methyl group or a t-butyl group is more preferred and a t-butyl group is even more preferred.

The aryl group represented by R₁ may be condensed and have a substituent. The substituent of the aryl group having a substituent may be the aforementioned substituent Z, and the substituent Z is preferably an alkyl group which may be substituted by a fluorine atom, an aryl group, a fluorine atom or a cyano group, more preferably an alkyl group. The aryl group represented by R₁ is preferably an aryl group having 6 to 30 carbon atoms, more preferably an aryl group having 6 to 18 carbon atoms. The aryl group having 6 to 18 carbon atoms is preferably an aryl group having 6 to 18 carbon atoms which may have an alkyl group having 1 to 6 carbon atoms that may be substituted by a fluorine atom, a fluorine atom or a cyano group, more preferably an aryl group having 6 to 18 carbon atoms which may have an alkyl group having 1 to 4 carbon atoms. Examples thereof include a phenyl group, a dimethylphenyl group, a biphenyl group, a terphenyl group, a naphthyl group, a methylnaphthyl group, a t-butylnaphthyl group, an anthranyl group, a phenanthryl group, a chrysenyl group, a cyanophenyl group, a trifluoromethylphenyl group, a fluorophenyl group and the like. Among them, a phenyl group, a dimethylphenyl group, a biphenyl group, a terphenyl group, a naphthyl group, a methylnaphthyl group, or a t-butylnaphthyl group is preferred and a phenyl group, a biphenyl group or a terphenyl group is more preferred.

The silyl group represented by R₁ may have a substituent. The substituent of the silyl group having a substituent may be the aforementioned substituent Z, and the substituent Z is preferably an alkyl group or a phenyl group, more preferably a phenyl group. The silyl group represented by R₁ is preferably a silyl group having 0 to 18 carbon atoms, more preferably a silyl group having 3 to 18 carbon atoms. The silyl group having 3 to 18 carbon atoms is preferably a silyl group having 3 to 18 carbon atoms substituted by an alkyl group having 1 to 6 carbon atoms or a phenyl group, more preferably a silyl group in which all of three hydrogen atoms are substituted by an alkyl group having 1 to 6 carbon atoms or a phenyl group, even more preferably a silyl group substituted by a phenyl group. Examples of the silyl group include a trimethylsilyl group, a triethylsilyl group, a t-butyldimethylsilyl group, a diethylisopropylsilyl group, a dimethylphenylsilyl group, a diphenylmethylsilyl group, a triphenylsilyl group and the like. Among them, a trimethylsilyl group, a dimethylphenylsilyl group or a triphenylsilyl group is preferred and a triphenylsilyl group is more preferred.

In a case in which R₁ is present in plural, each of a plurality of R₁'s may be the same as or different from every other R₁. In addition, the plurality of R₁'s may be combined together to form an aryl ring which may have the aforementioned substituent Z. The substituent Z is preferably an alkyl group or an aryl group, more preferably an alkyl group.

The aryl ring formed by combining a plurality of R₁'s together is preferably an aryl ring having 6 to 30 carbon atoms, more preferably an aryl ring having 6 to 14 carbon atoms, including carbon atoms substituted by the plurality of R₁'s. The formed ring is preferably any one of a benzene ring, a naphthalene ring and a phenanthrene ring, more preferably a benzene ring or a phenanthrene ring, even more preferably a benzene ring. Furthermore, the ring formed by a plurality of R₁'s may be present in a plural. For example, a plurality of R₁'s is combined together to form two benzene rings so that a phenanthrene ring is formed together with the benzene ring to which the plurality of R₁'s are substituted.

In terms of electric charge transporting performance and electric charge-associated stability, R₁ is preferably any one of an alkyl group, an aryl group that may have an alkyl group, a silyl group substituted by an alkyl group or a phenyl group, more preferably an aryl group having 6 to 18 carbon atoms which may have an alkyl group having 1 to 6 carbon atoms, more preferably an aryl group having 6 to 18 carbon atoms which may have an alkyl group having 1 to 4 carbon atoms.

Among them, R₁ is preferably a methyl group, a t-butyl group, a neopentyl group, a unsubstituted phenyl group, a phenyl group substituted by a cyano group, a fluorine atom or a trifluoromethyl group, a biphenyl group, a terphenyl group, a unsubstituted naphthyl group, a naphthyl group substituted by a methyl group or a t-butyl group, a triphenylsilyl group, a benzene ring or a phenanthrene ring formed by combining a plurality of alkyl groups or aryl groups together, more preferably a unsubstituted phenyl group, a biphenyl group, a terphenyl group or a benzene ring formed by combining a plurality of alkyl groups together, more preferably a unsubstituted phenyl group, a terphenyl group or a benzene ring formed by combining a plurality of alkyl groups together.

n1 is preferably an integer of 0 to 4, more preferably an integer of 0 to 3, even more preferably an integer of 0 to 2.

Specific examples and preferred examples of the aryl and silyl groups represented by R₂ to R₅ are the same as specific examples and preferred examples of the aryl and silyl groups represented by R₁.

Examples of the alkyl group represented by R₂ to R₅ include, in addition to examples of the alkyl group represented by R₁, a perfluoroalkyl alkyl group such as trifluoromethyl group. Among them, a methyl group, a trifluoromethyl group, an isopropyl group, a t-butyl group or a neopentyl group is preferred, a methyl group or a t-butyl group is more preferred, and a t-butyl group is even more preferred.

In terms of electric charge transporting performance and electric charge-associated stability, each of R₂ to R₅ is independently preferably any one of an alkyl group, an aryl group, a silyl group substituted by an alkyl group or a phenyl group, a cyano group and a fluorine atom, more preferably any one of an alkyl group having 1 to 6 carbon atoms, an aryl group having 6 to 18 carbon atoms, a silyl group having 3 to 18 carbon atoms substituted by an alkyl group having 1 to 6 carbon atoms or a phenyl group, a cyano group and a fluorine atom, even more preferably any one of an alkyl group having 1 to 4 carbon atoms, an aryl group having 6 to 12 carbon atoms, a silyl group having 3 to 18 carbon atoms substituted by an alkyl group having 1 to 6 carbon atoms or a phenyl group, a cyano group and a fluorine atom.

Among them, each of R₂ to R₅ is independently preferably any one of a methyl group, an isopropyl group, a t-butyl group, a neopentyl group, a trifluoromethyl group, a phenyl group, a dimethylphenyl group, a trimethylsilyl group, a triphenylsilyl group, a fluorine atom and a cyano group, more preferably a t-butyl group, a phenyl group, a trimethylsilyl group, a triphenylsilyl group and a cyano group, even more preferably any one of a t-butyl group, a phenyl group, a triphenylsilyl group and a cyano group.

Each of n2 to n5 is independently preferably an integer of 0 to 2, more preferably 0 or 1. In a case in which a substituent is incorporated into a carbazole structure, 3- and 6-positions of the carbazole structure are reactive positions and the substituent is preferably incorporated into these positions in terms of easy synthesis and improvement in chemical stability.

The compound represented by Formula (1) is more preferably represented by Formula (2).

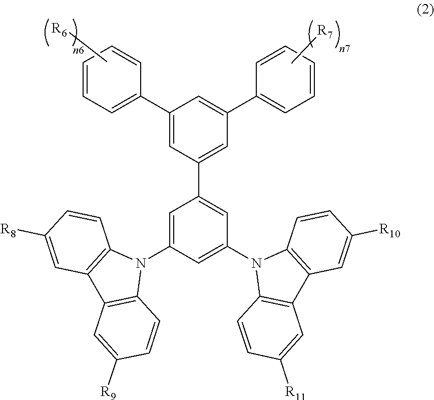

In Formula (2), each of R₆ and R₇ independently represents an alkyl group which may have a substituent Z, an aryl group which may have an alkyl group, a cyano group or a fluorine atom. In a case in which each of R₆ and R₇ is present in plural, each of the plurality of R₆'s and the plurality of R₇'s may be the same as or different from every other R₆ and R₇, respectively. In addition, each of a plurality of R₆'s and a plurality of R₇'s may be combined together to form an aryl ring that may have a substituent Z.

Each of n6 and n7 independently represents an integer of 0 to 5.

Each of R₈ to R₁₁ independently represents a hydrogen atom, an alkyl group which may have a substituent Z, an aryl group which may have an alkyl group, a silyl group which may have a substituent Z, a cyano group or a fluorine atom.

The substituent Z represents an alkyl group, an alkenyl group, an aryl group, an aromatic heterocyclic group, an alkoxy group, a phenoxy group, a fluorine atom, a silyl group, an amino group, a cyano group or a group formed by combination thereof and a plurality of substituents Z may be combined together to form an aryl group.

The alkyl group represented by R₆ and R₇ may have a substituent and may be saturated or unsaturated. The substituent of the alkyl group having a substituent may be the aforementioned substituent Z and the substituent Z is preferably a fluorine atom.

The alkyl group represented by R₆ and R₇ is preferably an alkyl group having 1 to 6 carbon atoms, more preferably an alkyl group having 1 to 4 carbon atoms. Specific examples and preferred examples of the alkyl group represented by R₆ and R₇ are the same as specific examples and preferred examples of the alkyl group represented by R₂ to R₅.

In the aryl group which may have an alkyl group represented by R₆ and R₇, the alkyl group is preferably an alkyl group having 1 to 6 carbon atoms, more preferably an alkyl group having 1 to 4 carbon atoms. Specific examples and preferred examples of the alkyl group are the same as specific examples and preferred examples of the alkyl group represented by R₂ to R₅.

In the aryl group which may have an alkyl group, represented by R₆ and R₇, the aryl group is preferably an aryl group having 6 to 18 carbon atoms, more preferably an aryl group having 6 to 12 carbon atoms. Examples of the aryl group include a phenyl group, a biphenyl group, a terphenyl group, a naphthyl group, an anthranyl group, a phenanthryl group, a chrysenyl group and the like. Among them, a phenyl group, a biphenyl group, a terphenyl group or a naphthyl group is preferred, and a phenyl group, a biphenyl group or a terphenyl group is more preferred.

The aryl group which may have an alkyl group represented by R₆ and R₇ is preferably an unsubstituted aryl group.

Examples of the aryl group which may have an alkyl group represented by R₆ and R₇ include a phenyl group, a dimethylphenyl group, a t-butylphenyl group, a biphenyl group, a terphenyl group, a naphthyl group, a methylnaphthyl group, a t-butylnaphthyl group, an anthranyl group, a phenanthryl group, a chrysenyl group and the like. Among them, a phenyl group, a t-butylphenyl group or a biphenyl group is preferred and a phenyl group is more preferred.

In a case in which each of R₆ and R₇ are present in plural, each of the plurality of R₆'s and the plurality of R₇'s may be the same as or different from every other R₆ and R₇, respectively. In addition, each of a plurality of R₆'s and a plurality of R₇'s may be combined together to form an aryl ring that may have a substituent Z. The substituent Z is preferably an alkyl group or an aryl group, more preferably an alkyl group.

The aryl group formed by combining a plurality of R₆'s and a plurality of R₇'s together is preferably an aryl ring having 6 to 30 carbon atoms, more preferably an aryl ring having 6 to 14 carbon atoms, even more preferably an aryl ring having 6 to 14 carbon atom which may have an alkyl group having 1 to 4 carbon atoms, including carbon atoms to which the plurality of R₆'s and the plurality of R₇'s are substituted. The formed ring is preferably any one of a benzene ring, a naphthalene ring and a phenanthrene ring, which may have an alkyl group having 1 to 4 carbon atoms, more preferably a benzene ring which may have an alkyl group having 1 to 4 carbon atoms, and examples thereof include a benzene ring, a benzene ring substituted by a t-butyl group and the like. Furthermore, the ring formed by combining a plurality of R₆'s and a plurality of R₇'s together may be present in plural. For example, each of a plurality of R₆'s or a plurality of R₇'s are combined together to form two benzene rings so that a phenanthrene ring is formed together with the benzene ring to which the plurality of R₆'s or the plurality of R₇'s are substituted.

In terms of electric charge transporting performance and electric charge-associated stability, R₆ and R₇ are preferably any one of an alkyl group having 1 to 6 carbon atoms, an aryl group having 6 to 18 carbon atoms which may have an alkyl group having 1 to 6 carbon atoms, a cyano group and a fluorine atom, more preferably any one of an alkyl group having 1 to 4 carbon atoms, an aryl group having 6 to 12 carbon atoms which may have an alkyl group having 1 to 4 carbon atoms, a cyano group and a fluorine atom. In terms of electric charge transporting performance and electric charge-associated stability, each of R₆ and R₇ independently preferably represents an alkyl group or an aryl group which may have an alkyl group.

Among them, each of R₆ and R₇ independently preferably represents a methyl group, a trifluoromethyl group, a t-butyl group, a unsubstituted phenyl group, a phenyl group substituted by a t-butyl group, a biphenyl group, a cyano group, a fluorine atom, a unsubstituted benzene ring formed by combining a plurality of alkyl groups together or a benzene ring substituted by a t-butyl group, more preferably a methyl group, a trifluoromethyl group, unsubstituted phenyl group, a cyano group, a fluorine atom, a unsubstituted benzene ring formed by combining a plurality of alkyl groups together or a benzene ring substituted by a t-butyl group, most preferably a unsubstituted phenyl group.

Each of n6 and n7 independently preferably represents an integer of 0 to 4, more preferably an integer of 0 to 2, even more preferably 0 or 1.

The alkyl group represented by R₈ to R₁₁ may have a substituent and may be saturated or unsaturated. The substituent of the alkyl group having a substituent may be the aforementioned substituent Z and the substituent Z is preferably a fluorine atom.

The alkyl group represented by R₈ to R₁₁ is preferably an alkyl group having 1 to 6 carbon atoms, more preferably an alkyl group having 1 to 4 carbon atoms. Specific examples and preferred examples of the alkyl group represented by R₈ to R₁₁ are the same as specific examples and preferred examples of the alkyl group represented by R₂ to R₅.

The aryl group which may have an alkyl group represented by R₈ to R₁₁ is preferably an aryl group having 6 to 18 carbon atoms which may have an alkyl group having 1 to 6 carbon atoms, more preferably an aryl group having 6 to 12 carbon atoms which may have an alkyl group having 1 to 4 carbon atoms.

Specific examples and preferred examples of the aryl group which may have the alkyl group represented by R₈ to R₁₁ are the same as specific examples and preferred examples of the aryl group which may have the alkyl group represented by R₆ and R₇.

The silyl group represented by R₈ to R₁₁ may have a substituent. The substituent of the silyl group having a substituent may be the aforementioned substituent Z and the substituent Z is preferably an alkyl group or a phenyl group, more preferably a phenyl group. The silyl group represented by R₈ to R₁₁ is preferably a silyl group having 3 to 18 carbon atoms, and specific examples and preferred examples of a silyl group having 3 to 18 carbon atoms represented by R₈ to R₁₁ are the same as specific examples and preferred examples of the silyl group having 3 to 18 carbon atoms of the silyl group represented by R₁.

In terms of electric charge transporting performance and electric charge-associated stability, each of R₈ to R₁₁ is independently preferably any one of a hydrogen atom, an alkyl group, an aryl group which may have an alkyl group, a silyl group substituted by an alkyl group or a phenyl group, a cyano group and a fluorine atom, more preferably any one of a hydrogen atom, an alkyl group having 1 to 6 carbon atoms, an aryl group having 6 to 18 carbon atoms which may have an alkyl group having 1 to 6 carbon atoms, a silyl group having 3 to 18 carbon atoms substituted by an alkyl group having 1 to 6 carbon atoms or a phenyl group, a cyano group and a fluorine atom, more preferably any one of a hydrogen atom, an alkyl group having 1 to 4 carbon atoms, an aryl group having 6 to 12 carbon atoms which may have an alkyl group having 1 to 4 carbon atoms, a silyl group having 3 to 18 carbon atoms substituted by an alkyl group having 1 to 6 carbon atoms or a phenyl group, a cyano group and a fluorine atom.

Among them, each of R₈ to R₁₁ is independently preferably any one of a hydrogen atom, a methyl group, an isopropyl group, a t-butyl group, a neopentyl group, a trifluoromethyl group, a phenyl group, a dimethylphenyl group, a trimethylsilyl group, a triphenylsilyl group, a fluorine atom and a cyano group, more preferably a hydrogen atom, a t-butyl group, a phenyl group, a trimethylsilyl group, a triphenylsilyl group and a cyano group, more preferably any one of a hydrogen atom, a t-butyl group, a phenyl group, a triphenylsilyl group and a cyano group.

The compound represented by Formula (1) or (2) is most preferably composed of only a carbon atom, a hydrogen atom and a nitrogen atom.

The compound represented by Formula (1) or (2) preferably has a glass transition temperature (Tg) of 80° C. to 400° C., more preferably 100° C. to 400° C., even more preferably 120° C. to 400° C.

In a case in which the compound of Formula (1) or (2) has a hydrogen atom, it contains an isotope (such as heavy hydrogen atom). In this case, all hydrogen atoms in the compound may be substituted by the isotope and some thereof may be a compound containing an isotope as a mixture.

Hereinafter, specific examples of the compound represented by Formula (1) or (2) are given and the present invention is not limited thereto.

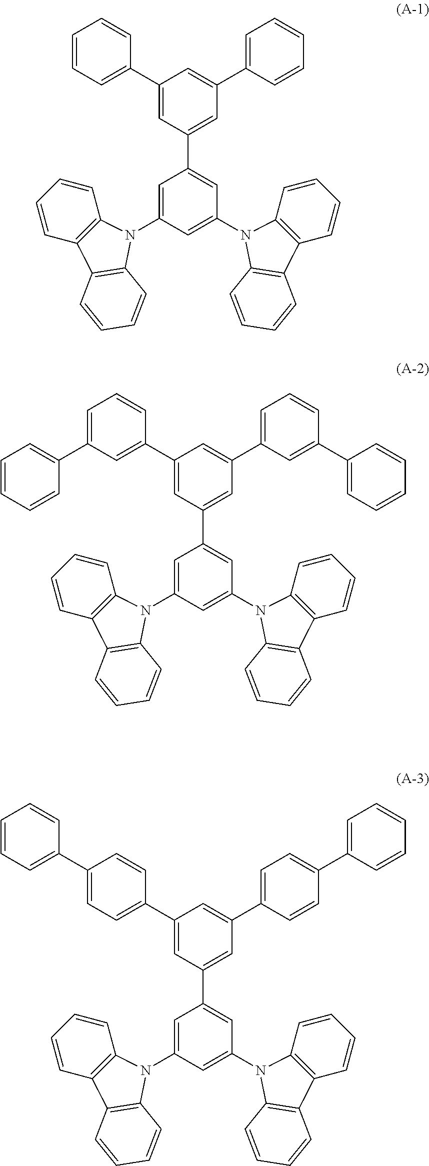 [SMILES: c1ccc(-c2cc(-c3ccccc3)cc(-c3cc(-n4c5ccccc5c5ccccc54)cc(-n4c5ccccc5c5ccccc54)c3)c2)cc1 ; c1ccc(-c2cccc(-c3cc(-c4cccc(-c5ccccc5)c4)cc(-c4cc(-n5c6ccccc6c6ccccc65)cc(-n5c6ccccc6c6ccccc65)c4)c3)c2)cc1 ; c1ccc(-c2ccc(-c3cc(-c4ccc(-c5ccccc5)cc4)cc(-c4cc(-n5c6ccccc6c6ccccc65)cc(-n5c6ccccc6c6ccccc65)c4)c3)cc2)cc1]

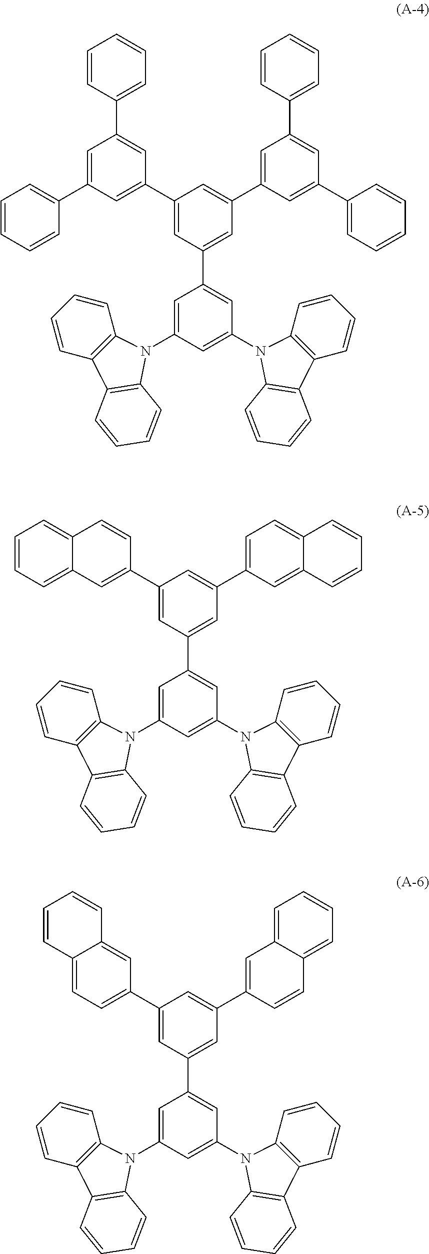 [SMILES: c1ccc(-c2cc(-c3ccccc3)cc(-c3cc(-c4cc(-c5ccccc5)cc(-c5ccccc5)c4)cc(-c4cc(-n5c6ccccc6c6ccccc65)cc(-n5c6ccccc6c6ccccc65)c4)c3)c2)cc1 ; c1ccc2cc(-c3cc(-c4cc(-n5c6ccccc6c6ccccc65)cc(-n5c6ccccc6c6ccccc65)c4)cc(-c4ccc5ccccc5c4)c3)ccc2c1 ; c1ccc2cc(-c3cc(-c4cc(-n5c6ccccc6c6ccccc65)cc(-n5c6ccccc6c6ccccc65)c4)cc(-c4ccc5ccccc5c4)c3)ccc2c1]

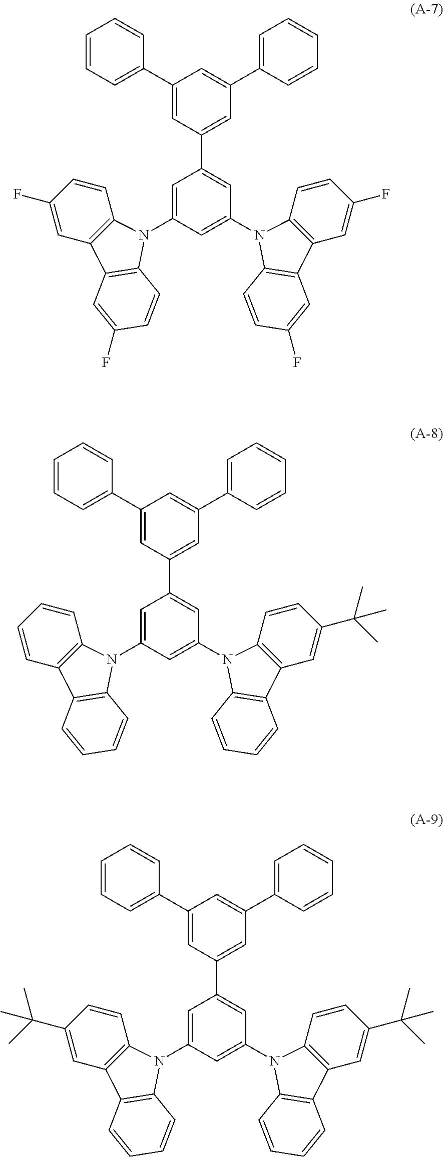

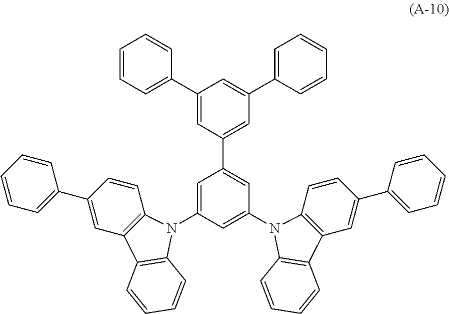 [SMILES: c1ccc(-c2cc(-c3ccccc3)cc(-c3cc(-n4c5ccccc5c5cc(-c6ccccc6)ccc54)cc(-n4c5ccccc5c5cc(-c6ccccc6)ccc54)c3)c2)cc1]

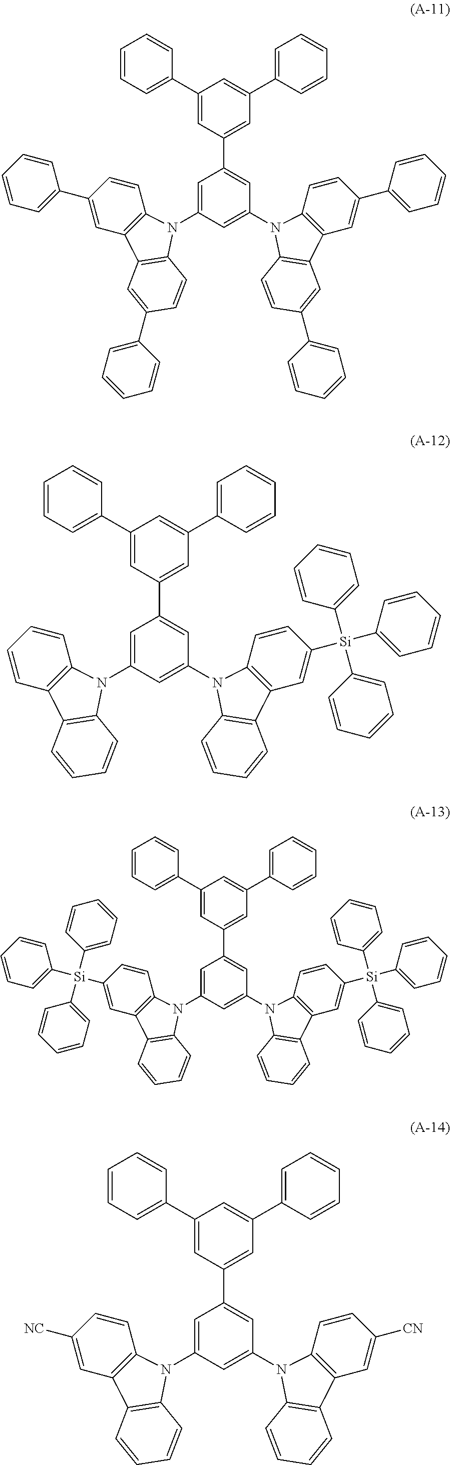

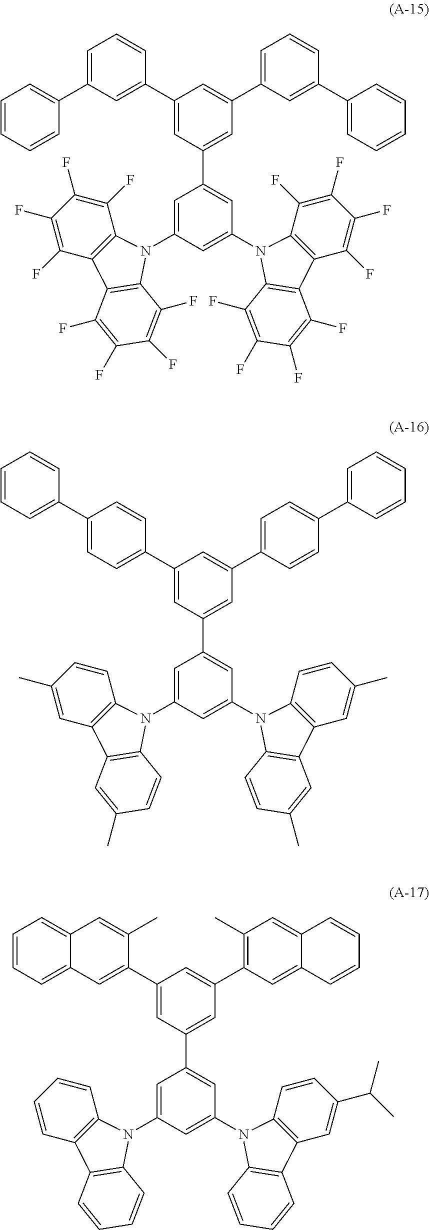 [SMILES: Fc1c(F)c(F)c2c(c1F)c1c(F)c(F)c(F)c(F)c1n2-c1cc(-c2cc(-c3cccc(-c4ccccc4)c3)cc(-c3cccc(-c4ccccc4)c3)c2)cc(-n2c3c(F)c(F)c(F)c(F)c3c3c(F)c(F)c(F)c(F)c32)c1 ; Cc1ccc2c(c1)c1cc(C)ccc1n2-c1cc(-c2cc(-c3ccc(-c4ccccc4)cc3)cc(-c3ccc(-c4ccccc4)cc3)c2)cc(-n2c3ccc(C)cc3c3cc(C)ccc32)c1 ; Cc1cc2ccccc2cc1-c1cc(-c2cc(-n3c4ccccc4c4ccccc43)cc(-n3c4ccccc4c4cc(C(C)C)ccc43)c2)cc(-c2cc3ccccc3cc2C)c1]

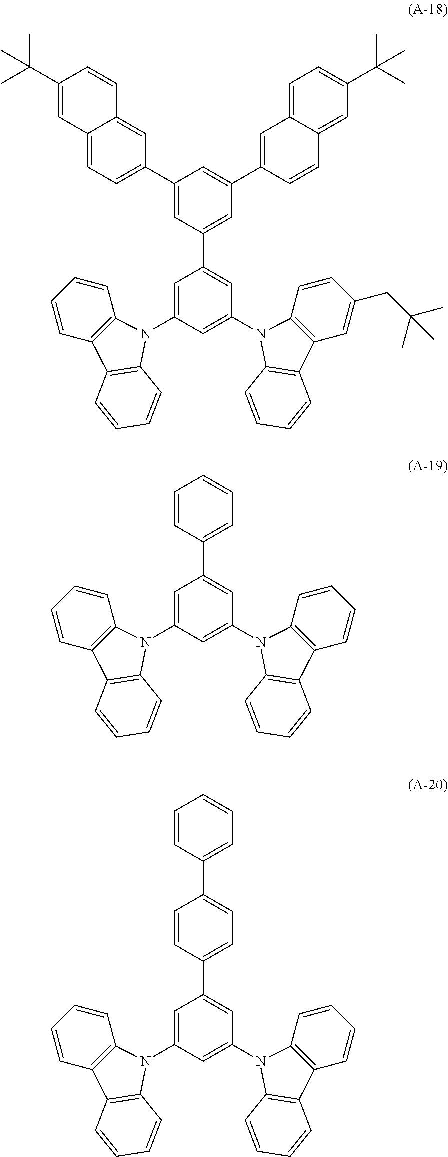 [SMILES: CC(C)(C)Cc1ccc2c(c1)c1ccccc1n2-c1cc(-c2cc(-c3ccc4cc(C(C)(C)C)ccc4c3)cc(-c3ccc4cc(C(C)(C)C)ccc4c3)c2)cc(-n2c3ccccc3c3ccccc32)c1 ; c1ccc(-c2cc(-n3c4ccccc4c4ccccc43)cc(-n3c4ccccc4c4ccccc43)c2)cc1 ; c1ccc(-c2ccc(-c3cc(-n4c5ccccc5c5ccccc54)cc(-n4c5ccccc5c5ccccc54)c3)cc2)cc1]

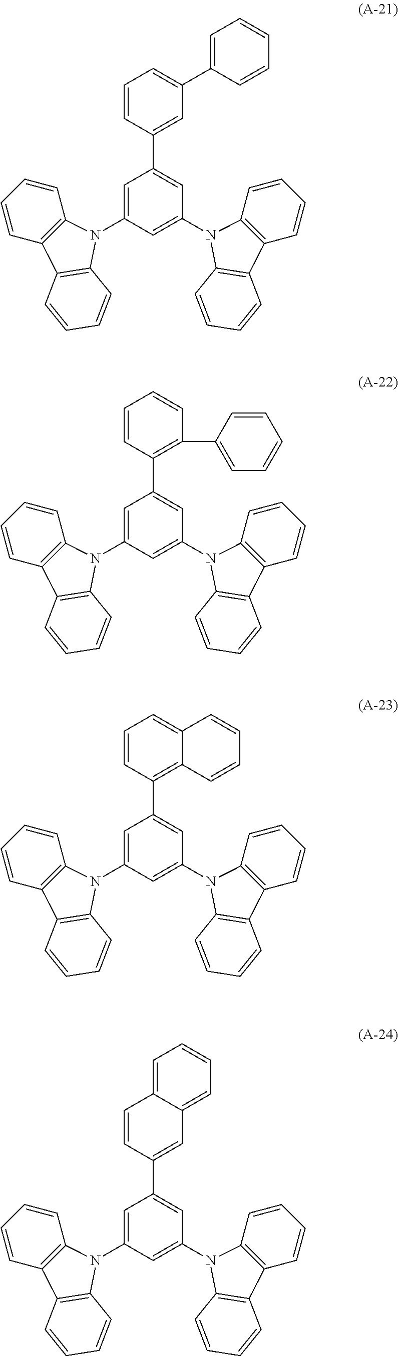 [SMILES: c1ccc(-c2cccc(-c3cc(-n4c5ccccc5c5ccccc54)cc(-n4c5ccccc5c5ccccc54)c3)c2)cc1 ; c1ccc(-c2ccccc2-c2cc(-n3c4ccccc4c4ccccc43)cc(-n3c4ccccc4c4ccccc43)c2)cc1 ; c1ccc2c(-c3cc(-n4c5ccccc5c5ccccc54)cc(-n4c5ccccc5c5ccccc54)c3)cccc2c1 ; c1ccc2cc(-c3cc(-n4c5ccccc5c5ccccc54)cc(-n4c5ccccc5c5ccccc54)c3)ccc2c1]

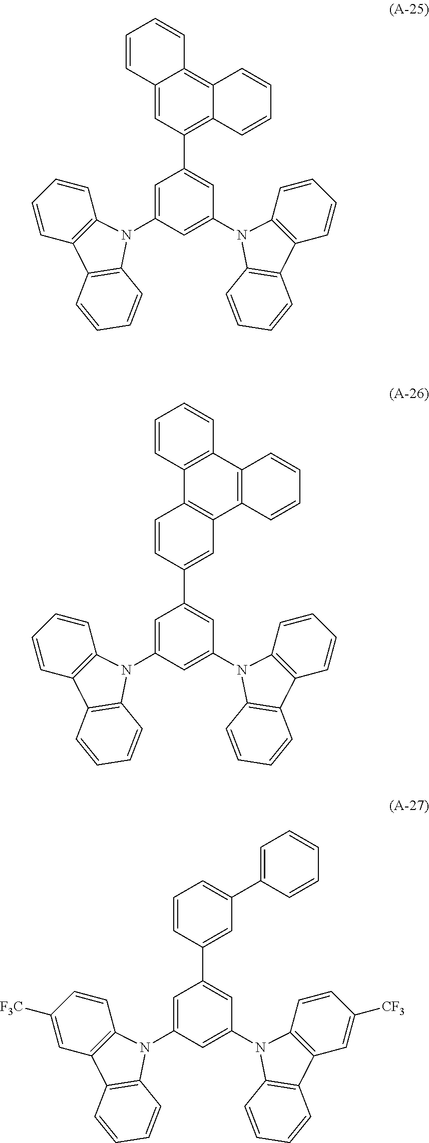 [SMILES: c1ccc2c(c1)cc(-c1cc(-n3c4ccccc4c4ccccc43)cc(-n3c4ccccc4c4ccccc43)c1)c1ccccc12 ; c1ccc2c(c1)c1ccccc1c1cc(-c3cc(-n4c5ccccc5c5ccccc54)cc(-n4c5ccccc5c5ccccc54)c3)ccc21 ; FC(F)(F)c1ccc2c(c1)c1ccccc1n2-c1cc(-c2cccc(-c3ccccc3)c2)cc(-n2c3ccccc3c3cc(C(F)(F)F)ccc32)c1]

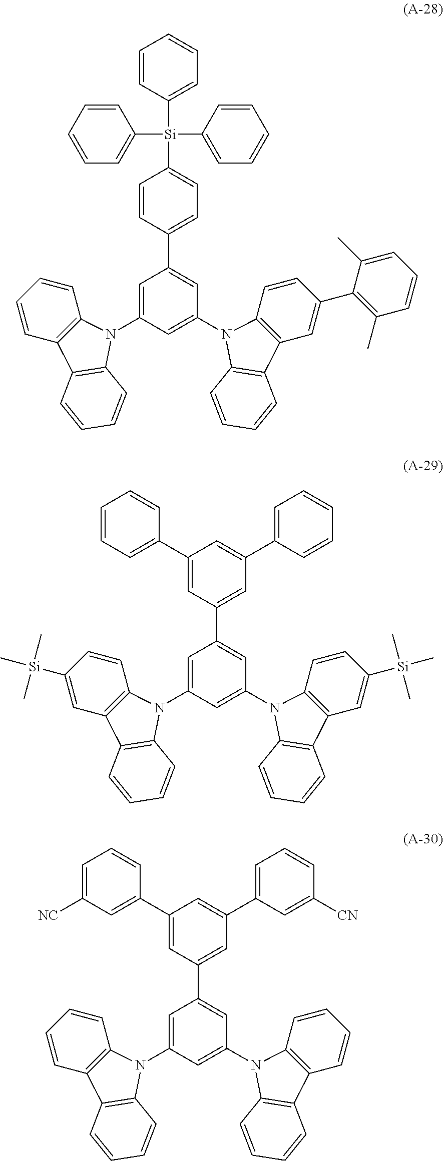

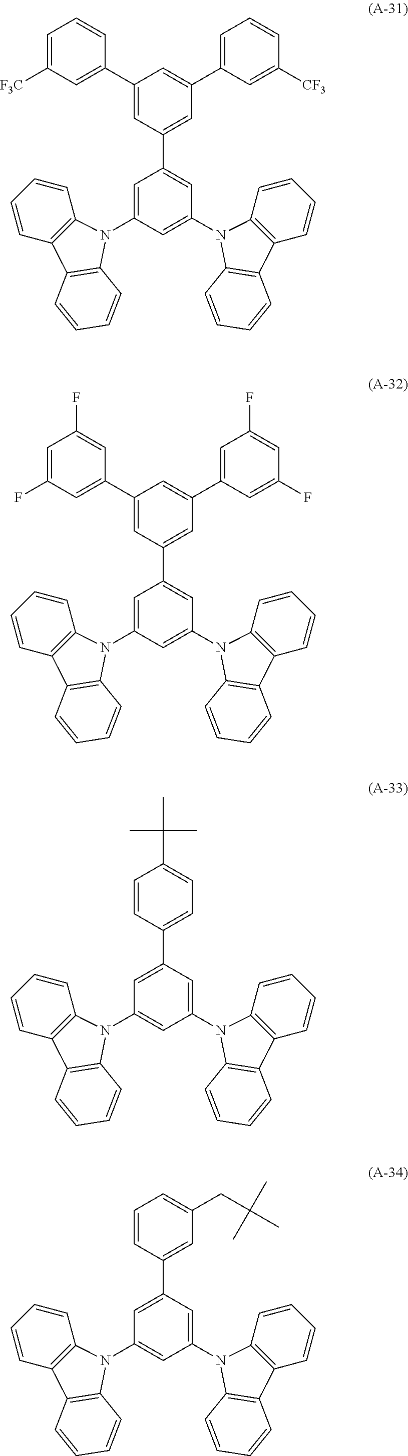 [SMILES: FC(F)(F)c1cccc(-c2cc(-c3cc(-n4c5ccccc5c5ccccc54)cc(-n4c5ccccc5c5ccccc54)c3)cc(-c3cccc(C(F)(F)F)c3)c2)c1 ; Fc1cc(F)cc(-c2cc(-c3cc(F)cc(F)c3)cc(-c3cc(-n4c5ccccc5c5ccccc54)cc(-n4c5ccccc5c5ccccc54)c3)c2)c1 ; CC(C)(C)c1ccc(-c2cc(-n3c4ccccc4c4ccccc43)cc(-n3c4ccccc4c4ccccc43)c2)cc1 ; CC(C)(C)Cc1cccc(-c2cc(-n3c4ccccc4c4ccccc43)cc(-n3c4ccccc4c4ccccc43)c2)c1]

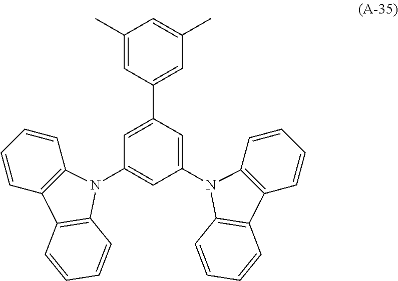 [SMILES: Cc1cc(C)cc(-c2cc(-n3c4ccccc4c4ccccc43)cc(-n3c4ccccc4c4ccccc43)c2)c1]

The compound exemplified as the compound represented by Formula (1) or (2) is synthesized based on WO 2004/074399 or the like. For example, the compound (A-1) may be synthesized by a method described in WO 2004/074399, page 52, line 22 to page 54, line 15.

In the present invention, use of the compound represented by Formula (1) or (2) is not limited and may be contained in any layer of the organic layer. The layer into which the compound represented by Formula (1) or (2) is incorporated is preferably one or more of a light emitting layer, a hole injection layer, a hole transporting layer, an electron transporting layer, an electron injection layer, an exciton block layer and an electric charge block layer.

In the present invention, in order to further suppress variation in chromaticity after driving at high temperatures, the compound represented by Formula (1) or (2) is preferably contained in the light emitting layer or any one adjacent layer to the light emitting layer, more preferably contained in the light emitting layer. In addition, the compound represented by Formula (1) or (2) may be contained in both the light emitting layer and the layer adjacent thereto.

When the compound represented by Formula (1) or (2) is contained in the light emitting layer, the compound represented by Formula (1) or (2) of the present invention is contained in an amount of 0.1 to 99% by mass, more preferably 1 to 95% by mass, even more preferably 10 to 95% by mass, with respect to the total mass of the light emitting layer. When the compound represented by Formula (1) or (2) is further contained, in addition to the light emitting layer, in the other layer, the compound is preferably contained in an amount of 70 to 100% by mass, more preferably 85 to 100% by mass, with respect to the total mass of the layer.

[Light Emitting Layer Containing Compound Represented by Formula (PQ-1) and Compound Represented by Formula (1) or (2)]

The present invention is also directed to a light emitting layer containing a compound represented by Formula (PQ-1) and a compound represented by Formula (1) or (2). The light emitting layer of the present invention may be used for an organic electroluminescence device.

[Composition Containing Compound Represented by Formula (PQ-1) and Compound Represented by Formula (1) or (2)]

The present invention is also directed to a composition containing a compound represented by Formula (PQ-1) and a compound represented by Formula (1) or (2).

In the composition of the present invention, the content of the compound represented by Formula (PQ-1) is preferably 1 to 40% by mass, more preferably 3 to 20% by mass, with respect to the total solid content of the composition.

In the composition of the present invention, the content of the compound represented by Formula (1) or (2) is preferably 50 to 97% by mass, more preferably 70 to 90% by mass, with respect to the total solid content of the composition.

The component which may be further contained in the composition of the present invention may be an organic or inorganic substance. Examples of useful organic substances include the host material, a fluorescent light emitting material, a phosphorescent light emitting material, a hydrocarbon substance. An organic layer of an organic electroluminescence device can be formed using the composition of the present invention by a dry-type film formation method such as a deposition method or a sputtering method, or a wet-type film formation method such as a transfer method or a printing method.

[Organic Electroluminescence Device]

The device of the present invention will be described in more detail.

The organic electroluminescence device of the present invention is an organic electroluminescence device that includes a pair of electrodes disposed on a substrate, and at least one organic layer including a light emitting layer disposed between the electrodes. The light emitting layer contains at least one compound represented by Formula (PQ-1) and any one of the at least one organic layer contains at least one compound represented by Formula (1).

In the organic electroluminescence device of the present invention, the light emitting layer is an organic layer and may further have a plurality of organic layers.

In terms of characteristics of the device, at least one electrode of the anode and cathode is preferably transparent or semitransparent.

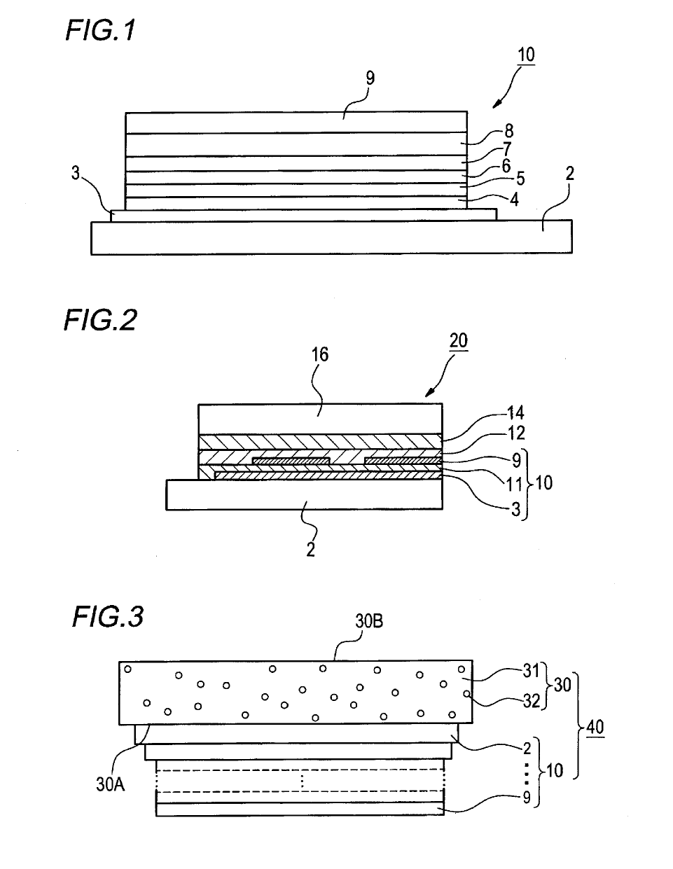

FIG. 1 illustrates an example of a layer configuration of the organic electroluminescence device. As shown in FIG. 1, the organic electroluminescence device 10 according to the present invention has a structure in which a light emitting layer 6 is interposed between an anode 3 and a cathode 9 on a substrate 2. Specially, a hole injection layer 4, a hole transporting layer 5, a light emitting layer 6, a hole block layer 7 and an electron transporting layer 8 are laminated in this order between the anode 3 and the cathode 9.

<Configuration of Organic Layer>

The configuration of layer constituting the organic layer is not particularly limited and may be suitably selected depending on application and purpose of the organic electroluminescence device. The organic layer is preferably formed on a transparent electrode or a semitransparent electrode. In this case, the organic layer is formed on the front surface or one surface of the transparent electrode or the semitransparent electrode.

The shape, size and thickness of organic layer are not particularly limited and can be suitably selected depending on the purpose.

Specific examples of the specific layer configuration are given below and the present invention is not limited thereto.

Anode/hole transporting layer/light emitting layer/electron transporting layer/cathode,

Anode/hole transporting layer/light emitting layer/block layer/electron transporting layer/cathode,

Anode/hole transporting layer/light emitting layer/block layer/electron transporting layer/electron injection layer/cathode,

Anode/hole injection layer/hole transporting layer/light emitting layer/electron transporting layer/electron injection layer/cathode

Anode/hole injection layer/hole transporting layer/light emitting layer/block layer/electron transporting layer/cathode,

Anode/hole injection layer/hole transporting layer/light emitting layer/block layer/electron transporting layer/electron injection layer/cathode.

The device configuration, substrate, cathode and anode of the organic electroluminescence device are for example described in the pamphlet of Japanese Patent Publication No. 2008-270736 in detail and the contents described in the pamphlet may be applied to the present invention.

<Substrate>

The substrate used in the present invention is preferably a substrate which does not scatter or decrease light emitted from an organic layer. The organic material preferably exhibits superior heat resistance, dimensional stability, solvent resistance, electrical insulating property and processability.

<Anode>

Any anode may be used so long as it serves as an electrode supplying holes into an organic layer and the shape, structure and size thereof are not particularly limited and may be suitably selected from known electrode material depending on the application and purpose of luminescence device. As mentioned above, the anode is commonly disposed as a transparent anode.

<Cathode>

Any cathode may be used so long as it serves as an electrode supplying electrons into the organic layer and the shape, structure and size thereof are not particularly limited and may be suitably selected from known electrode material depending on the application and purpose of luminescence device. As mentioned above, the anode is commonly disposed as a transparent anode.

The contents of the substrate, anode, cathode described in the paragraphs [0070] to [0089] of Japanese Patent Publication No. 2008-270736 may be applied to the present invention.

<Organic Layer>

The organic layer of the present invention will be described.

—Formation of Organic Layer—

In the organic electroluminescence device of the present invention, each organic layer may be preferably formed by a dry-type film formation method such as a deposition method or a sputtering method, or a solution coating process such as a transfer method, a printing method, a spin coating method, or a bar coating method. At least one layer of the organic layer is preferably formed by a solution coating process.

(Light Emitting Layer)

<Light Emitting Material>

The light emitting material of the present invention is preferably a compound represented by Formula (PQ-1).

The light emitting material of the light emitting layer is contained in an amount of 0.1% by mass to 50% by mass, with respect to the total mass of the compound constituting the light emitting layer in the light emitting layer. The content is preferably 1% by mass to 50% by mass, more preferably 2% by mass to 40% by mass, in terms of durability and external quantum efficiency.

The thickness of the light emitting layer is not particularly limited, and is generally preferably 2 nm to 500 nm, more preferably 3 nm to 200 nm, even more preferably 5 nm to 100 nm, in terms of external quantum efficiency.

In the device of the present invention, the light emitting layer may be composed of only a light emitting material and composed of a mixture of a host material and a light emitting material. The light emitting material may be a fluorescent light emitting material or a phosphorescent light emitting material. The dopant may be one or more types. The host material is preferably an electric charge transporting material. The host material may be one or more types. For example, the host material is composed of a combination of an electron transporting host material and a hole transporting host material. Furthermore, the light emitting layer may contain a material that does not have electric charge transporting property and does not emit light. In the device of the present invention, the light emitting layer preferably uses a compound represented by Formula (1) or (2) as a host material and a compound represented by Formula (PQ-1) as a light emitting material.

In addition, the light emitting layer may be a monolayer or a multilayer including two or more layers. When the light emitting layer is present in plural, the compound represented by Formula (1) or (2) and the compound represented by (PQ-1) may be also contained in the light emitting layer including two or more layers. In addition, respective light emitting layers may emit light having different colors.

<Host Material>

The host material used in the present invention is preferably a compound represented by Formula (1) or (2).

The compound represented by Formula (1) or (2) is a compound capable of transporting two electric charges of holes and electrons. By combining compound represented by Formula (1) or (2) with the compound represented by Formula (PQ-1), it is possible to prevent the balance between hole and electron transporting properties in the light emitting layer from being changed by exterior environment such as temperature or electric field. As a result, it is possible to improve driving durability although the compound has a carbazole group. Furthermore, it is possible to inhibit color variation after driving at a high temperature.

The host material used in the present invention may contain the following compound. Examples of the host material include pyrole, indole, carbazole (such as CBP(4,4′-di(9-carbazolyl)biphenyl)), azeindole, azecarbazole, triazole, oxazole, oxadiazole, pyrazole, imidazole, thiophene, polyarylalkane, pyrazolene, pyrazolone, phenylene diamine, arylamine, amino-substituted kalcone, styryl anthracene, fluorenone, hydrazone, stilbene, silazane, aromatic tertiary amine compounds, styrylamine compounds, porphyrin-based compounds, polysilane-based compounds, poly(N-vinylcarbazole), aniline-based copolymers, thiophene oligomers, conductive polymer oligomers such as polythiophene, organic silane, carbon films, pyridine, pyrimidine, triazine, imidazole, pyrazole, triazole, oxazole, oxadiazole, fluorenone, anthraquinodimethane, anthrone, diphenylquinone, thiopyran dioxide, carbodiimide, fluorenylidene methane, distyrylpyrazine, fluorine-substituted aromatic compounds, heterocyclic ring tetracarboxylic anhydrides such as naphthalene perylene, phthalocyanine, metal complexes or metal phthalocyanine of 8-quinolinol derivatives, various metal complexes and derivative thereof (may have a substituent or a condensed ring) represented by metal complexes having benzoxazole or benzothiazole as a ligand and the like.

In the light emitting layer of the present invention, the host material (also containing the compound represented by Formula (1) or (2)) having the least triplet excited energy (T₁ energy) higher than T₁ energy of the phosphorescent light emitting material is preferred in terms of color purity, light emitting efficiency and driving durability.

In addition, the content of host compound in the present invention is not particularly limited and is preferably 15% by mass to 98% by mass, with respect to the total mass of the compound constituting the light emitting layer, in terms of light emitting efficiency and driving voltage.

(Fluorescent Light Emitting Material)

Examples of the fluorescent light emitting material that can be used for the present invention include benzoxazole derivatives, benzoimidazole derivatives, benzothiazole derivatives, styrylbenzene derivatives, polyphenyl derivatives, diphenyl butadiene derivatives, tetraphenyl butadiene derivatives, naphthalimide derivatives, coumarin derivatives, condensed aromatic compounds, perinone derivatives, oxadiazole derivatives, oxazine derivatives, aldazine derivatives, pyrralidine derivatives, cyclopentadiene derivatives, bisstyryl anthracene derivatives, quinacridone derivatives, pyrrolopyridine derivatives, thiadiazolepyridine derivatives, cyclopentadiene derivatives, styrylamine derivatives, diketopyrrolopyrrole derivatives, aromatic dimethylidine compounds, various complexes represented by complexes of 8-quinolinol derivatives or complexes of pyrromethene derivatives, polymer compounds such as polythiophene, polyphenylene, polyphenylene vinylene, compounds such as organic silane derivatives and the like.

(Phosphorescent Light Emitting Material)

Examples of the phosphorescent light emitting material that can be used in the present invention include, in addition to the compound represented by Formula (PQ-1), phosphorescent light emitting compounds described in Patent Documents such as U.S. Pat. No. 6,303,238B1, U.S. Pat. No. 6,097,147, WO 00/57676, WO 00/70655, WO 01/08230, WO 01/39234A2, WO 01/41512A1, WO 02/02714A2, WO 02/15645A1, WO 02/44189A1, WO 05/19373A2, JP-A-2001-247859, JP-A-2002-302671, JP-A-2002-117978, JP-A-2003-133074, JP-A-2002-235076, JP-A-2003-123982, JP-A-2002-170684, EP1211257, JP-A-2002-226495, JP-A-2002-234894, JP-A-2001-247859, JP-A-2001-298470, JP-A-2002-173674, JP-A-2002-203678, JP-A-2002-203679, JP-A-2004-357791, JP-A-2006-256999, JP-A-2007-19462, JP-A-2007-84635, JP-A-2007-96259. Among them, more preferred light emitting materials include Ir complexes, Pt complexes, Cu complexes, Re complexes, W complexes, Rh complexes, Ru complexes, Pd complexes, Os complexes, Eu complexes, Tb complexes, Gd complexes, Dy complexes and Ce complexes. Particularly preferred light emitting materials include Ir complexes, Pt complexes, and Re complexes. Among them, Ir complexes, Pt complexes or Re complexes containing at least one coordination method of metal-carbon bonds, metal-nitrogen bonds, metal-oxygen bonds and metal-sulfur bonds are preferred. Furthermore, in terms of light emitting efficiency, driving durability and chromaticity, Ir complexes, Pt complexes or Re complexes containing multi-dentate ligands of three or more dentates are particularly preferred.

The content of the phosphorescent light emitting material that can be used in the present invention (compound represented by Formula (PQ-1) and/or phosphorescent light emitting material used in conjunction therewith) is 0.1% by mass to 50% by mass, more preferably 1% by mass to 40% by mass, most preferably 5% by mass to 30% by mass, with respect to the total mass of the light emitting layer. In particular, when the content is 5% by mass to 30% by mass, chromaticity of light emission of the organic electroluminescence device hardly depends on the concentration of added phosphorescent light emitting material.

Most preferably, the organic electroluminescence device of the present invention contains an amount of 5 to 30% by mass of the at least one of the compound (PQ-1) (Compound represented by Formula (PQ-1)), with respect to the total mass of the light emitting layer.

(Electric Charge Transporting Layer)

The electric charge transporting layer refers to a layer in which electric charges are moved when a voltage is applied to an organic electroluminescence device. Specific examples of the electric charge transporting layer include a hole injection layer, a hole transporting layer, an electron block layer, a light emitting layer, a hole block layer, an electron transporting layer, an electron injection layer and the like. Preferred examples include a hole injection layer, a hole transporting layer, an electron block layer and a light emitting layer. When the electric charge transporting layer formed by an application method is a hole injection layer, a hole transporting layer, an electron block layer or a light emitting layer, an organic electroluminescence device that is cheap and has high efficiency can be produced. In addition, the electric charge transporting layer is more preferably a hole injection layer, a hole transporting layer or an electron block layer.

—Hole Injection Layer, Hole Transporting Layer—

The hole injection layer and hole transporting layer are layers that have the ability of transporting holes from an anode or the side of the anode to a cathode side.

The hole injection layer preferably contains an electron accepting dopant. When the electron accepting dopant is contained in the hole injection layer, there are effects such as improvement of hole injection property, deterioration in driving voltage and improvement in efficiency.

Any organic or inorganic material may be used as the electron accepting dopant so long as it can extract electrons from a doped material and produce cations. Examples of the material include benzoquinone or derivatives thereof and metal oxides. Preferred are tetracyanoquinodimethane (TCNQ), tetrafluorotetracyanoquinodimethane (F₄-TCNQ) and molybdenum oxide.

The electron accepting dopant in the hole injection layer is preferably contained in an amount of 0.01% by mass to 50% by mass, more preferably 0.1% by mass to 40% by mass, even more preferably 0.5% by mass to 30% by mass, with respect to the total mass of the compound constituting the hole injection layer.

—Electron Injection Layer, Electron Transporting Layer—

The electron injection layer and the electron transporting layer are layers that have the ability of transporting electrons from the cathode or the side of the cathode to the anode side.

The electron injection layer preferably contains an electron donating dopant. When the electron donating dopant is contained in the electron injection layer, there are effects such as improvement of electron injection property, deterioration in driving voltage and improvement in efficiency.

Any organic or inorganic material may be used as the electron donating dopant so long as it can supply electrons to a doped material and produce radical anions. Examples of the electron donating dopant include tetrathiafulvalene (TTF), tetrathianaphthacene (TTT), lithium, cesium and the like.

The electron donating dopant in the electron injection layer is preferably contained in an amount of 0.1% by mass to 50% by mass, more preferably 0.1% by mass to 40% by mass, even more preferably 0.5% by mass to 30% by mass, with respect to the total mass of the compound constituting the electron injection layer.

Regarding the hole injection layer, the hole transporting layer, the electron injection layer and the electron transporting layer, the contents described in the paragraphs [0165] to [0167] of JP-A-2008-270736 may be applied to the present invention.

In the device of the present invention, the device containing an electron accepting dopant or an electron donating dopant exhibits improved external quantum efficiency than a device not containing them. The reason for this is not clear, but it is thought as follows. When an electron injection property or hole injection property is improved, the balance of electric charge in the light emitting layer is broken and light emission position varies. When the hole injection property is improved, electric charges are accumulated on the interface of the side of the cathode of the light emitting layer and a light emission ratio increases at the position, and when the electron injection property is improved, electric charges are accumulated on the interface of the side of the anode of the light emitting layer and a light emission ratio increases at the position. In devices containing no electron accepting dopant or electron donating dopant exhibits great variation of this light emission position, efficiency is greatly deteriorated due to inactivation of excitons caused by the hole block layer and the electron block layer, while devices containing an electron accepting dopant or an electron donating dopant do not exhibit great variation in light emission position and maintain efficiencies. As a result, it is thought that the relative value of external quantum efficiency.

—Hole Block Layer—

The hole block layer is a layer which prevents holes transported from the side of the anode to the light emitting layer from being escaped into the cathode. In the present invention, a hole block layer may be mounted as an organic layer adjacent to the light emitting layer in the side of the cathode.

Examples of the organic compound constituting the hole block layer include aluminum complexes such as aluminum (III) bis(2-methyl-8-quinolinato)4-phenylphenolate (simply referred to “BAlq”)), triazole derivatives, phenanthroline derivatives such as 2,9-dimethyl-4,7-diphenyl-1,10-phenanthroline (simply referred to “BCP”)), and the like.

The thickness of the hole block layer is preferably 1 nm to 500 nm, more preferably 5 nm to 200 nm, even more preferably 10 nm to 100 nm.

The hole block layer may be a monolayer made of one or more of the aforementioned materials, or a multilayer structure including a plurality of layers which have identical or different compositions.

—Electron Block Layer—

The electron block layer is a layer which prevents electrons transported from the cathode side to the light emitting layer from being escaped into the side of the anode. In the present invention, the electron block layer may be mounted as an organic layer adjacent to the light emitting layer in the side of the anode.

Examples of the organic compound constituting the electron block layer include those of the aforementioned examples of the hole transporting material.

The thickness of the electron block layer is preferably 1 nm to 500 nm, more preferably 5 nm to 200 nm, even more preferably 10 nm to 100 nm.

The electron block layer may be a monolayer made of one or more of the aforementioned materials, or a multilayer structure including two or more layers which have identical or different compositions.

<Protective Layer>

In the present invention, the organic EL device may be protected with a protective layer.

Regarding the protective layer, the content described in the paragraphs of [0169] to [0170] of JP-A-2008-270736 may be applied to the present invention.

<Sealing Container>

The device of the present invention may be sealed using a sealing container.

Regarding the sealing container, the content described in the paragraph of [0171] of JP-A-2008-270736 may be applied to the present invention.

(Driving)

The organic electroluminescence device of the present invention emits light by applying a direct voltage (may further include an alternating component, if necessary) between an anode and a cathode (commonly, 2 volt to 15 volt), or direct current.

The driving method of the organic electroluminescence device of the present invention may use driving methods described in the specifications of JP-A-2-148687, JP-A-6-301355, JP-A-5-29080, JP-A-7-134558, JP-A-8-234685, JP-A-8-241047, Japanese Patent No. 2784615, U.S. Pat. No. 5,828,429, and U.S. Pat. No. 6,023,308.

The luminescence device of the present invention can improve light extraction efficiency through various known methods. For example, light extraction efficiency and external quantum efficiency can be improved by processing the shape of substrate surface (for example, formation of fine roughness patterns), controlling refraction of substrate⋅ITO layer⋅organic layer, and controlling the thickness of substrate⋅ITO layer⋅organic layer.

The external quantum efficiency of the luminescence device of the present invention is preferably 15% to 30%. As the value of external quantum efficiency, a maximum external quantum efficiency when a device is driven at 80° C., or an external quantum efficiency of about 100 to 1000 cd/m² when a device is driven at 80° C. may be used.

The luminescence device of the present invention may be so-called a “top-emission type” in which light is emitted from the side of the anode.

The organic EL device of the present invention may have a resonator structure. For example, the organic EL device has a multi-layer film mirror including a plurality of laminated films having different refractive indexes, a transparent or semitransparent electrode, a light emitting layer and a metal electrode disposed on a transparent substrate. The light emitted from the light emitting layer is resonated by repeatedly being reflected between the multi-layer film mirror and a metal electrode as a reflection plate.

In more preferred embodiments, a transparent or semitransparent electrode and a metal electrode are served as reflection plates on a transparent substrate and light emitted from the light emitting layer is resonated by repeatedly being reflected therebetween.

To form the resonance structure, light passage length determined by effective refractive index of two reflection plates, refractive indexes of respective layers between the reflection plates and the thickness is adjusted to the most preferred value. In the first embodiment, the calculation equation is described in the specification of JP-A-9-180883. In the second embodiment, the calculation equation is described in the specification of JP-A-2004-127795.

(Use of the Luminescence Device of the Present Invention)

The luminescence device of the present invention may be preferably used for light emission apparatuses, pexels, display devices, displays, backlights, electrophotography, illumination light sources, recording light sources, exposure light sources, reading light sources, covers, signboards, interiors, optical communications and the like. In particular, the luminescence device is preferably used for devices which are driven in regions with high light emitting luminance intensity such as illumination apparatuses and display apparatuses.

(Light Emission Apparatus)

Then, the light emission apparatus of the present invention will be described with reference to FIG. 2.

The light emission apparatus of the present invention uses an organic electroluminescence device.

FIG. 2 is a sectional view schematically illustrating an example of a light emission apparatus of the present invention.

The light emission apparatus 20 of FIG. 2 includes a substrate (support substrate) 2, an organic electroluminescence device 10 and a sealing container 16.

The organic electroluminescence device 10 includes an anode (first electrode) 3, an organic layer 11, and a cathode (second electrode) 9 laminated on a substrate 2. In addition, the protective layer 12 is laminated on the cathode 9, and a sealing container 16 is mounted via an adhesive layer 14 on the protective layer 12. Furthermore, the part, barrier and insulating layer of respective electrodes 3 and 9 are omitted.

Here, a photosetting adhesive such as an epoxy resin or a thermosetting adhesive may be used as the adhesive layer 14 and, for example, a thermosetting adhesive sheet may be also used.

The use of the light emission apparatus of the present invention is not particularly limited and example thereof include illumination apparatuses as well as display apparatuses such as TVs, personal computers, cellular phones and electron papers.

(Illumination Apparatus)

Then, an illumination apparatus according to an embodiment of the present invention will be described with reference to FIG. 3.

FIG. 3 is a sectional view schematically illustrating an example of an illumination apparatus according to one embodiment of the present invention.

As shown in FIG. 3, the illumination apparatus 40 according to the embodiment of the present invention includes the aforementioned organic EL device 10 and a light scattering member 30. More specifically, the illumination apparatus 40 has a structure in which the substrate of the organic EL device 10 contacts the light scattering member 30.

Any material may be used as the light scattering member 30 so long as it can scatter light. As shown in FIG. 3, particles 32 are dispersed on a transparent substrate 31. Preferably, the transparent substrate 31 is for example a glass substrate. The particle 32 is preferably a transparent resin particle. The glass substrate and the transparent resin particle may be selected from known materials. Such an illumination apparatus 40 scatters the incident light through the light scattering member 30 and emits the scattered light from a light emission surface 30B as an illumination light, when light emitted from an organic electroluminescence device 10 is incident on a light incident surface 30A of the light scattering member 30.

### EXAMPLES

Hereinafter, the present invention will be described in detail with reference to examples in more detail and is not limited to the following specific examples.

The compound represented by Formula (1) or (2) used in Example is synthesized in accordance with WO 2004/074399 or the like. For example, the compound (A-1) is synthesized in accordance with the method described in WO 2004/074399, 52 pages 22 lines to 54 pages 15 lines. The compound represented by Formula (PQ-1) was synthesized in accordance with Japanese Patent No. 3929689. For example, FR-2 was synthesized in accordance with a method described in Japanese Patent No. 3929689, the paragraphs of [0054] to [0055] (page 18, lines 1 to 13).

Furthermore, all organic materials used in this example were sublimation-purified and analyzed by high-performance liquid chromatography (Tosoh TSKgel ODS-100Z), and materials having 99.9% or higher of an absorption intensity area ratio at 254 nm were used.

### Example 1-1

A glass substrate (produced by GEOMATEC CO., LTD, surface resistance: 10 Ω/□) having an indium tin oxide (ITO) film with a thickness of 0.5 mm and 2.5 cm square was incorporated into a cleaning container, ultrasonic cleaned in 2-propanol and treated with UV-ozone for 30 minutes. The following organic layers were sequentially deposited on the transparent anode (ITO film) by a vacuum deposition method.
First layer: CuPc (copper phthalocyanine): thickness 10 nm
Second layer: NPD (N,N′-di-α-naphthyl-N,N′-diphenyl)-benzidine): thickness 20 nm
Third layer: FR-1 (5% by mass), A-1 (95% by mass): thickness 30 nm
Fourth layer: BAlq: thickness 30 nm

Lithium fluoride was deposited to a thickness of 0.2 nm thereon and metal aluminum was then deposited to a thickness of 70 nm thereon to obtain a cathode.

The obtained laminate was incorporated into a grove box replaced with an argon gas without contacting an air, sealed using a sealing can made of stainless steel and a UV curable adhesive (XNR5516HV, produced by Nagase Chiba Co., Ltd.) to obtain an organic electroluminescence device of Example 1-1.

### Examples 1-2 to 1-29 and Comparative Examples 1-1 to 1-15

Organic electroluminescence devices of Examples 1-2 to 1-29 and Comparative Examples 1-1 to 1-15 were obtained in the same manner as in Example 1-1 except that a material constituting the third layer in Example 1-1 was changed into the material shown in the following Table 1. In Table 1, for evaluation of variation in chromaticity, the symbol “<” means a sign of inequality, and, for example, “<0.005” means that variation in chromaticity is lower than 0.005.

TABLE 1

| | | | External | | | |
|---|---|---|---|---|---|---|
| | | | quantum | | | |
| | | | efficiency | Durability | Variation | Variation in |
| | | | (relative | (relative | in voltage | chromaticity |
| | Host | Dopant | value) | value) | ΔV | (Δx, Δy) |
| Ex. 1-1 | A-1 | FR-1 | 15 | 240 | 0.8 | (<0.005, <0.005) |
| Ex. 1-2 | A-1 | FR-2 | 15 | 250 | 0.9 | (<0.005, <0.005) |
| Ex. 1-3 | A-1 | FR-5 | 15 | 220 | 1.0 | (<0.005, <0.005) |
| Ex. 1-4 | A-1 | FR-8 | 16 | 310 | 0.8 | (<0.005, <0.005) |
| Ex. 1-5 | A-1 | FR-11 | 14 | 180 | 1.1 | (<0.005, <0.005) |
| Ex. 1-6 | A-1 | FR-17 | 14 | 170 | 1.3 | (<0.005, <0.005) |
| Ex. 1-7 | A-1 | FR-22 | 14 | 180 | 1.0 | (<0.005, <0.005) |
| Ex. 1-8 | A-1 | FR-29 | 15 | 200 | 1.0 | (<0.005, <0.005) |
| Ex. 1-9 | A-4 | FR-1 | 15 | 260 | 0.8 | (<0.005, <0.005) |
| Ex. 1-10 | A-4 | FR-2 | 15 | 220 | 0.9 | (<0.005, <0.005) |
| Ex. 1-11 | A-4 | FR-6 | 14 | 170 | 1.2 | (<0.005, <0.005) |
| Ex. 1-12 | A-4 | FR-38 | 15 | 180 | 1.1 | (<0.005, <0.005) |
| Ex. 1-13 | A-9 | FR-1 | 16 | 220 | 0.9 | (<0.005. <0.005) |
| Ex. 1-14 | A-9 | FR-2 | 15 | 200 | 1.0 | (<0.005, <0.005) |
| Ex. 1-15 | A-9 | FR-9 | 15 | 190 | 1.1 | (<0.005, <0.005) |
| Ex. 1-16 | A-11 | FR-1 | 15 | 230 | 0.9 | (<0.005, <0.005) |
| Ex. 1-17 | A-11 | FR-2 | 14 | 220 | 0.9 | (<0.005, <0.005) |
| Ex. 1-18 | A-11 | FR-8 | 16 | 280 | 0.8 | (<0.005, <0.005) |
| Ex. 1-19 | A-11 | FR-29 | 14 | 220 | 0.8 | (<0.005, <0.005) |
| Ex. 1-20 | A-13 | FR-2 | 15 | 150 | 1.0 | (<0.005, <0.005) |
| Ex. 1-21 | A-13 | FR-7 | 15 | 160 | 1.0 | (<0.005, <0.005) |
| Ex. 1-22 | A-14 | FR-1 | 15 | 230 | 0.8 | (<0.005, <0.005) |
| Ex. 1-23 | A-14 | FR-8 | 16 | 210 | 0.8 | (<0.005, <0.005) |
| Ex. 1-24 | A-19 | FR-2 | 15 | 190 | 1.0 | (<0.005, <0.005) |
| Ex. 1-25 | A-19 | FR-30 | 15 | 220 | 1.0 | (<0.005, <0.005) |
| Ex. 1-26 | A-20 | FR-2 | 15 | 210 | 0.9 | (<0.005, <0.005) |
| Ex. 1-27 | A-20 | FR-3 | 14 | 200 | 1.0 | (<0.005, <0.005) |
| Ex. 1-28 | A-23 | FR-1 | 14 | 220 | 1.0 | (<0.005, <0.005) |
| Ex. 1-29 | A-23 | FR-8 | 15 | 210 | 0.9 | (<0.005, <0.005) |
| Comp. Ex. 1-1 | H-1 | E-1 | 10 | 100 | 2.1 | (0.02, 0.01) |
| Comp. Ex. 1-2 | H-1 | E-2 | 7 | 30 | 2.3 | (0.01, 0.01) |
| Comp. Ex. 1-3 | H-2 | E-1 | 9 | 60 | 2.0 | (<0.005, 0.02) |
| Comp. Ex. 1-4 | H-2 | E-2 | 7 | >10 | 2.5 | (0.01, 0.03) |
| Comp. Ex. 1-5 | H-1 | FR-5 | 11 | 90 | 1.9 | (<0.005, 0.02) |
| Comp. Ex. 1-6 | H-2 | FR-8 | 11 | 120 | 2.0 | (<0.005, 0.02) |
| Comp. Ex. 1-7 | A-1 | E-1 | 11 | 90 | 1.8 | (0.01, 0.03) |
| Comp. Ex. 1-8 | A-4 | E-1 | 10 | 90 | 1.9 | (0.01, 0.02) |
| Comp. Ex. 1-9 | A-9 | E-2 | 9 | 30 | 2.4 | (0.01, 0.02) |
| Comp. Ex. 1-10 | A-1 | E-3 | 9 | 40 | 2.7 | (0.01, 0.02) |
| Comp. Ex. 1-11 | BAlq | FR-8 | 10 | 90 | 2.4 | (<0.005, 0.02) |
| Comp. Ex. 1-12 | H-3 | FR-8 | 12 | 120 | 2.2 | (<0.005, 0.02) |
| Comp. Ex. 1-13 | H-4 | FR-8 | 12 | 80 | 2.3 | (<0.005, 0.02) |
| Comp. Ex. 1-14 | CBP | FR-8 | 11 | 70 | 2.3 | (<0.005, 0.02) |
| Comp. Ex. 1-15 | A-1 | Ir(ppy)₃ | 15 | 260 | 2.3 | (0.01, 0.02) |

### Example 2-1

A glass substrate (produced by GEOMATEC CO., LTD, surface resistance: 10 Ω/□) having an indium tin oxide (ITO) film with a thickness of 0.5 mm and 2.5 cm square was incorporated into a cleaning container, ultrasonic cleaned in 2-propanol and treated with UV-ozone for 30 minutes. The following organic layers were sequentially deposited on the transparent anode (ITO film) by a vacuum deposition method.
First layer: CuPc (copper phthalocyanine): thickness10 nm
Second layer: NPD (N,N′-di-α-naphthyl-N,N′-diphenyl)-benzidine): thickness 30 nm
Third layer: A-1: thickness 5 nm
Fourth layer: FR-1 (5% by mass), A-1 (95% by mass): thickness 30 nm
Fifth layer: A-1: thickness 3 nm
Sixth layer: BAlq: thickness 30 nm

Lithium fluoride was deposited to a thickness of 0.2 nm thereon and metal aluminum was then deposited to a thickness of 70 nm thereon to obtain a cathode.

The obtained laminate was incorporated into a grove box replaced with an argon gas without contacting an air, sealed using a sealing can made of stainless steel and a UV curable adhesive (XNR5516HV, produced by Nagase Chiba Co., Ltd.) to obtain an organic electroluminescence device of Example 2-1.

### Examples 2-2 to 2-5 and Comparative Examples 2-1 to 2-5

Organic electroluminescence devices of Examples 2-2 to 2-5 and Comparative Examples 2-1 to 2-5 were obtained in the same manner as in Example 2-1 except that FR-1 used for the fourth layer, and A-1 used for the third, fourth and fifth layers in Example 2-1 were changed into the materials shown in the following Table 2. In Table 2, for evaluation of variation in chromaticity, the symbol “<” means a sign of inequality, and, for example, “<0.005” means that variation in chromaticity is lower than 0.005.

TABLE 2

| | | | External | | | |
|---|---|---|---|---|---|---|
| | | | quantum | | Variation | |
| | Host/ | | efficiency | Durability | in | Variation in |
| | adjcent | | (relative | (relative | voltage | chromaticity |
| | layers | Dopant | value) | value) | ΔV | (Δx, Δy) |
| Ex. 2-1 | A-1 | FR-1 | 15 | 230 | 1.0 | (<0.005, <0.005) |
| Ex. 2-2 | A-4 | FR-2 | 14 | 220 | 0.9 | (<0.005, <0.005) |
| Ex. 2-3 | A-4 | FR-5 | 16 | 210 | 1.0 | (<0.005, <0.005) |
| Ex. 2-4 | A-11 | FR-8 | 15 | 250 | 0.9 | (<0.005, <0.005) |
| Ex. 2-5 | A-14 | FR-11 | 13 | 170 | 1.1 | (<0.005, <0.005) |
| Comp. Ex. 2-1 | H-1 | E-1 | 10 | 100 | 1.9 | (0.01, 0.02) |
| Comp. Ex. 2-2 | H-2 | FR-2 | 9 | 80 | 2.0 | (<0.005, 0.02) |
| Comp. Ex. 2-3 | A-1 | E-1 | 10 | 120 | 1.8 | (0.01, 0.03) |
| Comp. Ex. 2-4 | A-1 | E-2 | 8 | 30 | 2.2 | (0.01, 0.03) |
| Comp. Ex. 2-5 | A-1 | E-3 | 8 | 30 | 2.4 | (0.01, 0.02) |

### Example 3-1

A glass substrate (produced by GEOMATEC CO., LTD, surface resistance: 10 Ω/□) having an indium tin oxide (ITO) film with a thickness of 0.5 mm and 2.5 cm square was incorporated into a cleaning container, ultrasonic cleaned in 2-propanol and treated with UV-ozone for 30 minutes. A PEDOT (poly(3,4-ethylenedioxythiophene))/PSS(polystyrenesulfonic acid) aqueous solution (BaytronP (standard product)) was spin-coated (4000 rpm, 60 seconds) thereon and dried at 120° C. for 10 minutes to form a hole transporting layer (thickness 150 nm).

A toluene solution containing 1% by mass of a compound A-1 and 0.05% by mass of FR-2 was spin-coated (2000 rpm, 60 seconds) thereon to form a light emitting layer (thickness 50 nm). BAlq [bis-(2-methyl-8-quinolinate)-4-(phenylphenolate)aluminum] was deposited to 40 nm by a vacuum deposition method to obtain an electron transporting layer, lithium fluoride and metal aluminum were sequentially deposited to 0.2 nm and 150 nm, respectively, to obtain a cathode. The obtained structure was incorporated into a grove box replaced with an argon gas without contacting an air, sealed using a sealing can made of stainless steel and a UV curable adhesive (XNR5516HV, produced by Nagase Chiba Co., Ltd.) to obtain an organic electroluminescence device of Example 3-1.

### Examples 3-2 to 3-4 and Comparative Examples 3-1 to 3-5

Organic electroluminescence devices of Examples 3-2 to 3-4 and Comparative Examples 3-1 to 3-5 were obtained in the same manner as in Example 3-1 except that the materials constituting the light emitting layer in Example 3-1 were changed into the materials shown in the following Table 3. In Table 3, for evaluation of variation in chromaticity, the symbol “<” means a sign of inequality, and, for example, “<0.005” means that variation in chromaticity is lower than 0.005.

TABLE 3

| | | | External | | | |
|---|---|---|---|---|---|---|
| | | | quantum | | | |
| | | | efficiency | Durability | Variation | Variation in |
| | | | (relative | (relative | in voltage | chromaticity |
| | Host | Dopant | value) | value) | ΔV | (Δx, Δy) |
| Ex. 3-1 | A-1 | FR-2 | 14 | 220 | 1.0 | (<0.005, <0.005) |
| Ex. 3-2 | A-4 | FR-8 | 15 | 270 | 1.0 | (<0.005, <0.005) |
| Ex. 3-3 | A-9 | FR-5 | 14 | 210 | 1.1 | (<0.005, <0.005) |
| Ex. 3-4 | A-13 | FR-1 | 13 | 210 | 1.1 | (<0.005, <0.005) |
| Comp. Ex. 3-1 | H-1 | E-1 | 10 | 100 | 2.1 | (0.01, 0.03) |
| Comp. Ex. 3-2 | H-2 | FR-2 | 8 | 90 | 1.9 | (0.01, 0.02) |
| Comp. Ex. 3-3 | A-1 | E-1 | 10 | 110 | 2.0 | (0.01, 0.03) |
| Comp. Ex. 3-4 | A-1 | E-2 | 8 | 110 | 2.2 | (0.01, 0.03) |
| Comp. Ex. 3-5 | A-1 | E-3 | 8 | 100 | 2.3 | (0.01, 0.03) |

### Example 4-1

A glass substrate (produced by GEOMATEC CO., LTD, surface resistance: 10 Ω/□) having an indium tin oxide (ITO) film with a thickness of 0.5 mm and 2.5 cm square was incorporated into a cleaning container, ultrasonic cleaned in 2-propanol and treated with UV-ozone for 30 minutes. The following organic layers were sequentially deposited on the transparent anode (ITO film) by a vacuum deposition method.
First layer: CuPc (copper phthalocyanine): thickness 10 nm
Second layer: NPD (N,N′-di-α-naphthyl-N,N′-diphenyl)-benzidine): thickness 20 nm
Third layer: FR-1 (5% by mass), A-1 (95% by mass): thickness 30 nm
Fourth layer: BAlq: thickness 10 nm
Fifth layer: BCP (99% by mass), Li (1% by mass): thickness 30 nm

Lithium fluoride was deposited to a thickness of 0.2 nm thereon and metal aluminum was then deposited to a thickness of 70 nm thereon to obtain a cathode.

The obtained laminate was incorporated into a grove box replaced with an argon gas without contacting an air, sealed using a sealing can made of stainless steel and a UV curable adhesive (XNR5516HV, produced by Nagase Chiba Co., Ltd.) to obtain an organic electroluminescence device of Example 4-1.

### Examples 4-2 to 4-4 and Comparative Examples 4-1 to 4-9

Organic electroluminescence devices of Examples 4-2 to 4-4 and Comparative Examples 4-1 to 4-9 were obtained in the same manner as in Example 4-1 except that FR-1 and A-1 used for the third layer in Example 4-1 were changed into materials shown in the following Table 4. In Table 4, for evaluation of variation in chromaticity, the symbol “<” means a sign of inequality, and, for example, “<0.005” means that variation in chromaticity is lower than 0.005.

TABLE 4

| | | | External | | | |
|---|---|---|---|---|---|---|
| | | | quantum | | Variation | |
| | | | efficiency | Durability | in | Variation in |
| | | | (relative | (relative | voltage | chromaticity |
| | Host | Dopant | value) | value) | ΔV | (Δx, Δy) |
| Ex. 4-1 | A-1 | FR-1 | 21 | 200 | 0.9 | (<0.005, <0.005) |
| Ex. 4-2 | A-4 | FR-2 | 22 | 210 | 0.9 | (<0.005, <0.005) |
| Ex. 4-3 | A-9 | FR-5 | 19 | 210 | 1.0 | (<0.005, <0.005) |
| Ex. 4-4 | A-11 | FR-8 | 24 | 240 | 0.8 | (<0.005, <0.005) |
| Comp. Ex. 4-1 | H-1 | E-1 | 10 | 100 | 2.0 | (0.01, 0.03) |
| Comp. Ex. 4-2 | H-2 | E-2 | 7 | 50 | 2.4 | (0.01, 0.03) |
| Comp. Ex. 4-3 | A-1 | E-1 | 11 | 130 | 1.9 | (0.01, 0.03) |
| Comp. Ex. 4-4 | H-1 | FR-1 | 11 | 130 | 1.9 | (0.01, 0.02) |
| Comp. Ex. 4-5 | A-1 | E-3 | 9 | 90 | 2.1 | (0.01, 0.03) |
| Comp. Ex. 4-6 | BAlq | FR-2 | 8 | 90 | 2.1 | (0.01, 0.03) |
| Comp. Ex. 4-7 | H-3 | FR-2 | 9 | 160 | 2.3 | (0.01, 0.03) |
| Comp. Ex. 4-8 | H-4 | FR-2 | 10 | 100 | 2.1 | (0.01, 0.03) |
| Comp. Ex. 4-9 | CBP | FR-2 | 9 | 80 | 2.1 | (0.01, 0.03) |

### Example 5-1

A glass substrate (produced by GEOMATEC CO., LTD, surface resistance: 10 Ω/□) having an indium tin oxide (ITO) film with a thickness of 0.5 mm and 2.5 cm square was incorporated into a cleaning container, ultrasonic cleaned in 2-propanol and treated with UV-ozone for 30 minutes. The following organic layers were sequentially deposited on the transparent anode (ITO film) by a vacuum deposition method.
First layer: 2-TNATA (99.7% by mass), F4-TCNQ (0.3% by mass): thickness 50 nm
Second layer: NPD (N,N′-di-α-naphthyl-N,N′-diphenyl)-benzidine): thickness 10 nm
Third layer: FR-1 (5% by mass), A-1 (95% by mass): thickness 30 nm
Fourth layer: BAlq: thickness 10 nm

Lithium fluoride was deposited to a thickness of 0.2 nm thereon and metal aluminum was then deposited to a thickness of 70 nm thereon to obtain a cathode.

The obtained laminate was incorporated into a grove box replaced with an argon gas without contacting an air, sealed using a sealing can made of stainless steel and a UV curable adhesive (XNR5516HV, produced by Nagase Chiba Co., Ltd.) to obtain an organic electroluminescence device of Example 5-1.

### Examples 5-2 to 5-4 and Comparative Examples 5-1 to 5-9

Organic electroluminescence devices of Examples 5-2 to 5-4 and Comparative Examples 5-1 to 5-9 were obtained in the same manner as in Example 5-1 except that FR-1 and A-1 used for the third layer in Example 5-1 were changed into materials shown in the following Table 5. In Table 5, for evaluation of variation in chromaticity, the symbol “<” means a sign of inequality, and, for example, “<0.005” means that variation in chromaticity is lower than 0.005.

TABLE 5

| | | | External | | | |
|---|---|---|---|---|---|---|
| | | | quantum | | Variation | |
| | | | efficiency | Durability | in | Variation in |
| | | | (relative | (relative | voltage | chromaticity |
| | Host | Dopant | value) | value) | ΔV | (Δx, Δy) |
| Ex. 5-1 | A-1 | FR-1 | 21 | 210 | 1.0 | (<0.005, <0.005) |
| Ex. 5-2 | A-4 | FR-8 | 23 | 230 | 0.9 | (<0.005, <0.005) |
| Ex. 5-3 | A-9 | FR-5 | 18 | 200 | 1.1 | (<0.005, <0.005) |
| Ex. 5-4 | A-11 | FR-2 | 20 | 200 | 1.0 | (<0.005, <0.005) |
| Comp. Ex. 5-1 | H-1 | E-1 | 10 | 100 | 2.0 | (0.01, 0.02) |
| Comp. Ex. 5-2 | H-2 | E-2 | 8 | 30 | 2.2 | (0.01, 0.03) |
| Comp. Ex. 5-3 | A-1 | E-1 | 10 | 120 | 1.9 | (0.01, 0.02) |
| Comp. Ex. 5-4 | H-1 | FR-1 | 10 | 90 | 2.0 | (<0.005, 0.02) |
| Comp. Ex. 5-5 | A-1 | E-3 | 8 | 100 | 2.0 | (0.01, 0.02) |
| Comp. Ex. 5-6 | BAlq | FR-1 | 10 | 100 | 2.0 | (0.01, 0.02) |
| Comp. Ex. 5-7 | H-3 | FR-1 | 12 | 150 | 2.3 | (0.01, 0.02) |
| Comp. Ex. 5-8 | H-4 | FR-1 | 11 | 110 | 2.2 | (0.01, 0.02) |
| Comp. Ex. 5-9 | CBP | FR-1 | 12 | 80 | 2.1 | (0.01, 0.02) |

(Evaluation of Performance of Organic Electroluminescence Device)

The performance of respective devices thus obtained was evaluated as follows.

(a) External Quantum Efficiency During Driving at High Temperature

Direct voltage was applied to respective devices at 80° C. in a thermostat using Source Measure Unit 2400 produced by Toyo Corporation to emit light, and the luminance intensity was measured using BM-8 produced by Topcon Corporation as a luminance intensity meter. Luminescent spectra and luminescent wavelengths were measured using a spectrum analyzer, PMA-11 produced by Hamamatsu Photonics K.K. Based on these values, external quantum efficiency at a luminance intensity of 1000 cd/m² was calculated by a luminance intensity conversion method, the value of Comparative Example 1-1 in Table 1, the value of Comparative Example 2-1 in Table 2, the value of Comparative Example 3-1 in Table 3, the value of Comparative Example 4-1 in Table 4, the value of Comparative Example 5-1 in Table 5 were set at 10 respectively and external quantum efficiencies were expressed as relative values in respective tables. The external quantum efficiency is preferably superior, as the value thereof increases.

(b) Durability During Driving at High Temperature

Respective devices were subjected to emitting light at 80° C. in a thermostat by applying direct voltage thereto such that luminance intensity became 5000 cd/m², the time taken until luminance intensity became 4000 cd/m² was set as an indicator of driving durability, the value of Comparative Example 1-1 in Table 1, the value of Comparative Example 2-1 in Table 2, the value of Comparative Example 3-1 in Table 3, the value of Comparative Example 4-1 in Table 4, the value of Comparative Example 5-1 in Table 5 were set to 100 respectively and durabilities were expressed as relative values with respect to these values in respective tables. The external quantum efficiency is preferably superior as the value thereof increases.

(c) Variation in Voltage After Driving at High Temperature

A difference between a DC voltage applied to each device in a 80° C. thermostat such that luminance intensity became 5000 cd/m² and a voltage applied thereto when luminance intensity became 4000 cd/m² after a DC voltage was continuously applied thereto, was set as an indicator of variation in voltage after driving at a high temperature and durabilities were expressed as a variation in voltage ΔV(V). The variation in voltage ΔV is preferably superior, as the value thereof decreases.

(d) Variation in Chromaticity After Driving at High Temperatures

Differences (Δx, Δy) in values x and y between chromaticity applied to each device in a 80° C. thermostat such that luminance intensity became 5000 cd/m², and chromaticity applied thereto when luminance intensity became 4000 cd/m² after a DC voltage was continuously applied thereto, was set as an indicator of variation in chromaticity after driving at a high temperature and the value was expressed as (Δx, Δy). The variation in chromaticity is preferably excellent, as the value thereof decreases.

As can be seen from the results of Tables 1 to 5, the device of the present invention using a host material containing a carbazole group represented by Formula (1) or (2) and a specific iridium complex represented by Formula (PQ-1) exhibits excellent external quantum efficiency and durability during driving at high temperatures, and excellent variation in voltage and variation in chromaticity after driving at a high temperature, as compared to the device of Comparative Example, and, in particular, exhibits considerably superior durability during driving at a high temperature.

The reason that the light emitting material and the host material of the present invention improve device performance after driving at high temperatures and, in particular, durability is not clear, but is thought as follows. As compared to room temperature, a device was driven at a high temperature, the film state may be readily changed and device defects readily occur. This behavior is thought to be observed in a material with a low molecular weight having a low glass transition temperature, materials that are readily crystallized due to large symmetricity and intermolecular interaction. In addition, in iridium complex-based phosphorescent materials, production of decomposer and quencher caused by isolation of the ligand which is a fate of the complex material is considered to deteriorate device performance. This deposition reaction also worsens with driving at high temperatures. In the present invention, variation in film state can be decreased by using a host material that has a large molecular weight and does not readily cause crystallization, and the area around iridium that is a central metal can be stereoscopically increased in volume and stability of complex can be improved by changing phenylisoquinoline into phenylquinoline as the ligand of the light emitting material. As a result, device performance is thought to be considerably improved due to these reasons.

Light emission apparatuses, display apparatuses and illumination apparatuses necessarily momentarily emit light with high luminance intensity based on high current density in respective pixels. In this case, the luminescence device of the present invention is advantageously used since it is designed such that it exhibits a high light emitting efficiency.

In addition, the device of the present invention exhibits superior light emitting efficiency or durability although used under high temperature conditions such as vehicle-mounting application and is thus preferably used for light emission apparatuses, display apparatuses and illumination apparatuses.

The structure of compounds used in Examples and Comparative Examples is shown as follows.

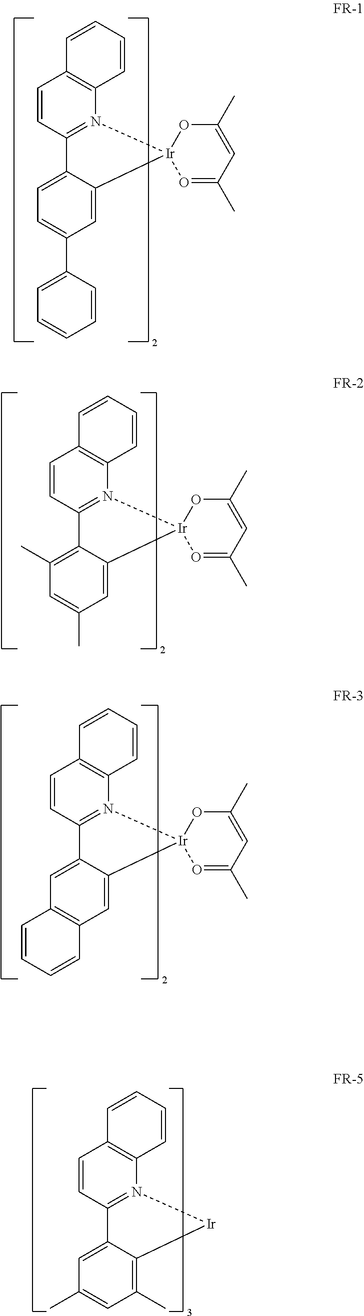

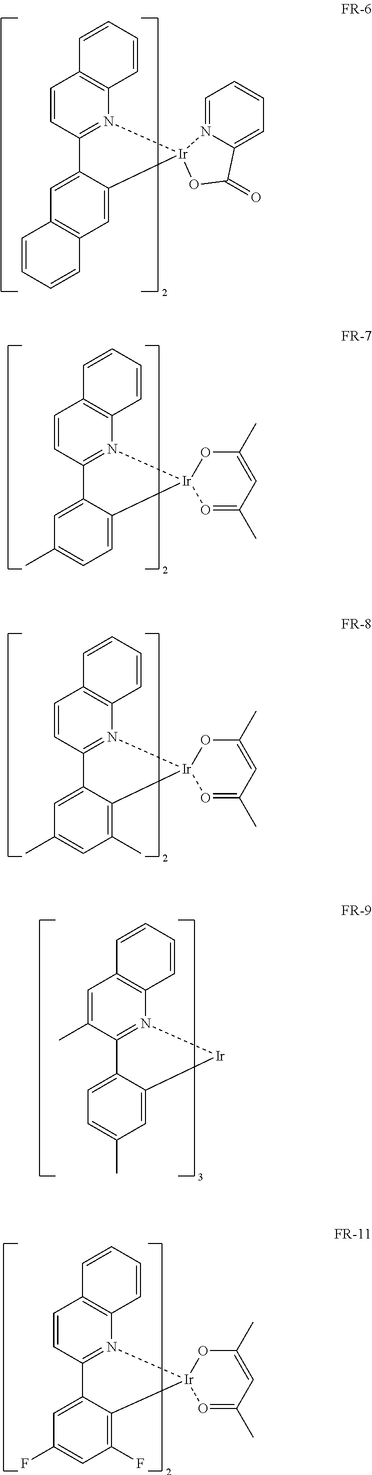

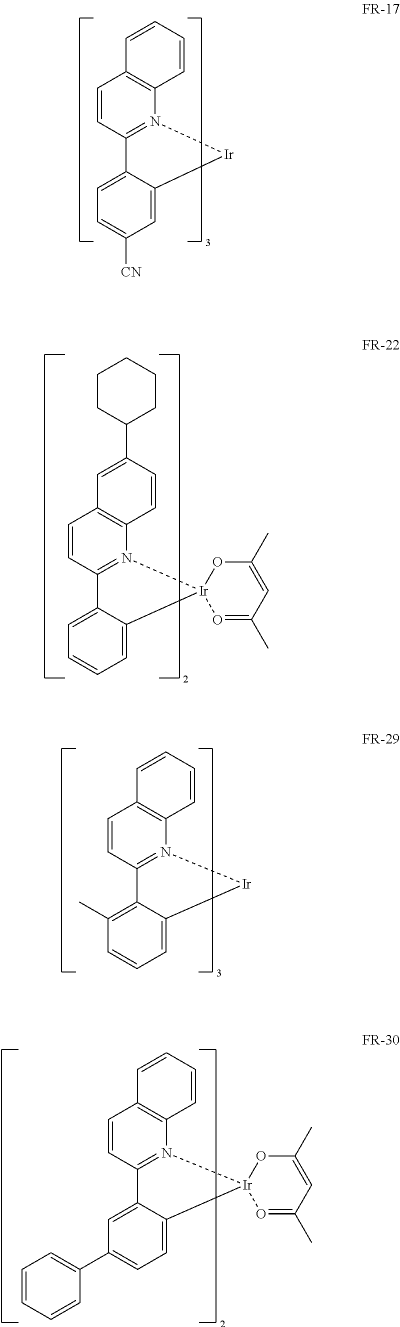

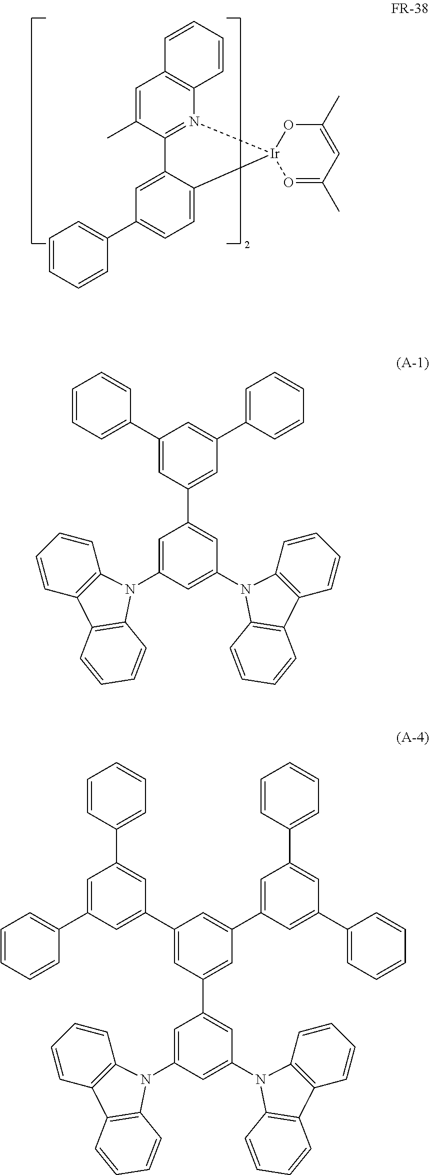

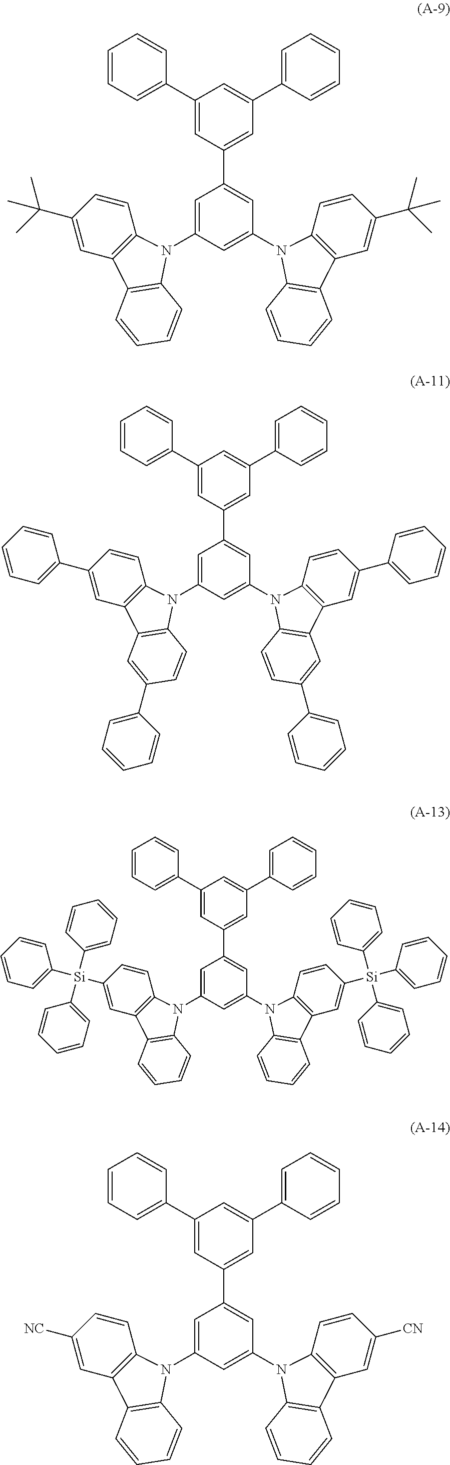 [SMILES: CC(C)(C)c1ccc2c(c1)c1ccccc1n2-c1cc(-c2cc(-c3ccccc3)cc(-c3ccccc3)c2)cc(-n2c3ccccc3c3cc(C(C)(C)C)ccc32)c1 ; c1ccc(-c2cc(-c3ccccc3)cc(-c3cc(-n4c5ccc(-c6ccccc6)cc5c5cc(-c6ccccc6)ccc54)cc(-n4c5ccc(-c6ccccc6)cc5c5cc(-c6ccccc6)ccc54)c3)c2)cc1 ; c1ccc(-c2cc(-c3ccccc3)cc(-c3cc(-n4c5ccccc5c5cc([Si](c6ccccc6)(c6ccccc6)c6ccccc6)ccc54)cc(-n4c5ccccc5c5cc([Si](c6ccccc6)(c6ccccc6)c6ccccc6)ccc54)c3)c2)cc1 ; N#Cc1ccc2c(c1)c1ccccc1n2-c1cc(-c2cc(-c3ccccc3)cc(-c3ccccc3)c2)cc(-n2c3ccccc3c3cc(C#N)ccc32)c1]

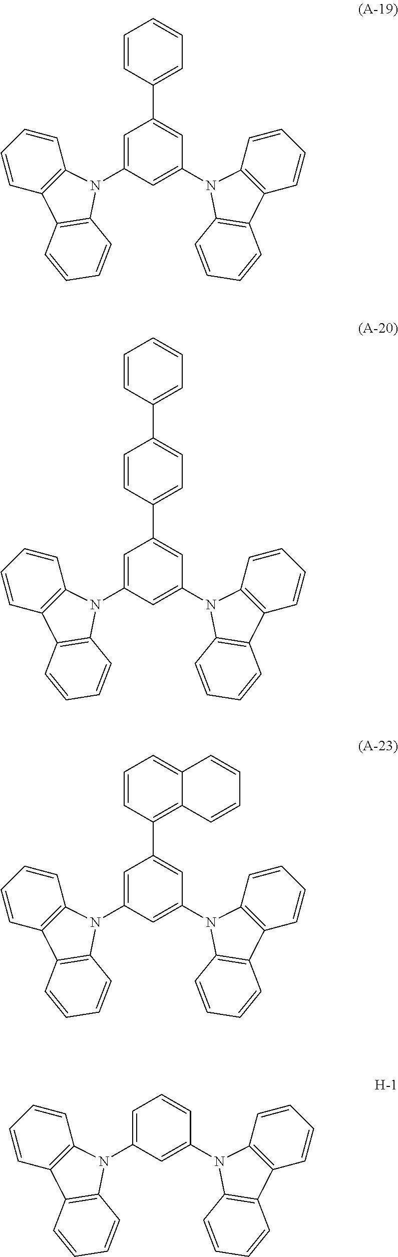 [SMILES: c1ccc(-c2cc(-n3c4ccccc4c4ccccc43)cc(-n3c4ccccc4c4ccccc43)c2)cc1 ; c1ccc(-c2ccc(-c3cc(-n4c5ccccc5c5ccccc54)cc(-n4c5ccccc5c5ccccc54)c3)cc2)cc1 ; c1ccc2c(-c3cc(-n4c5ccccc5c5ccccc54)cc(-n4c5ccccc5c5ccccc54)c3)cccc2c1 ; c1cc(-n2c3ccccc3c3ccccc32)cc(-n2c3ccccc3c3ccccc32)c1]

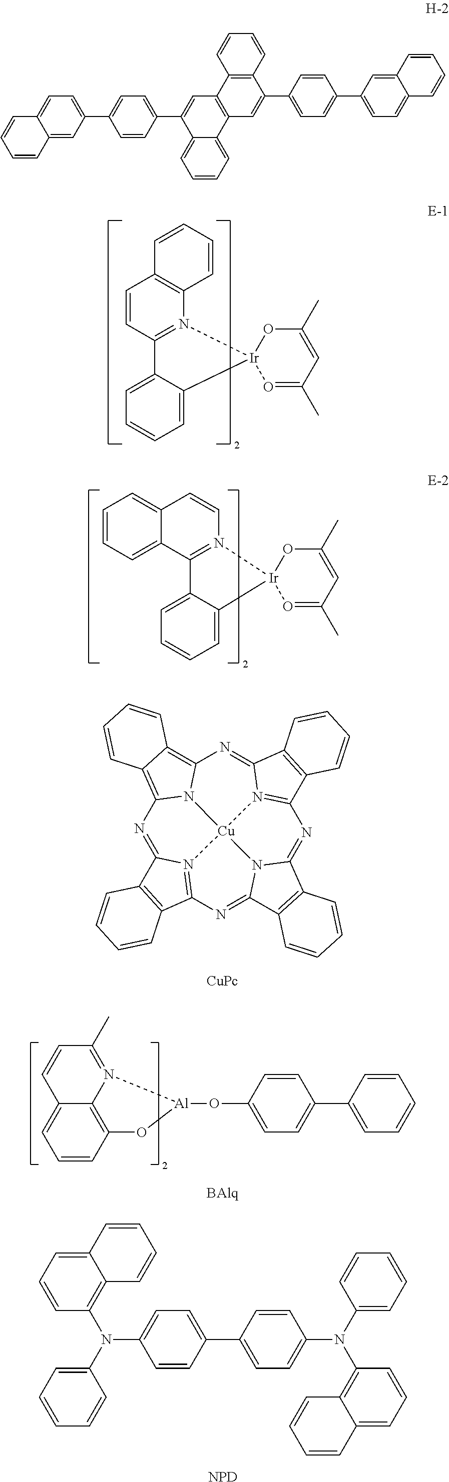

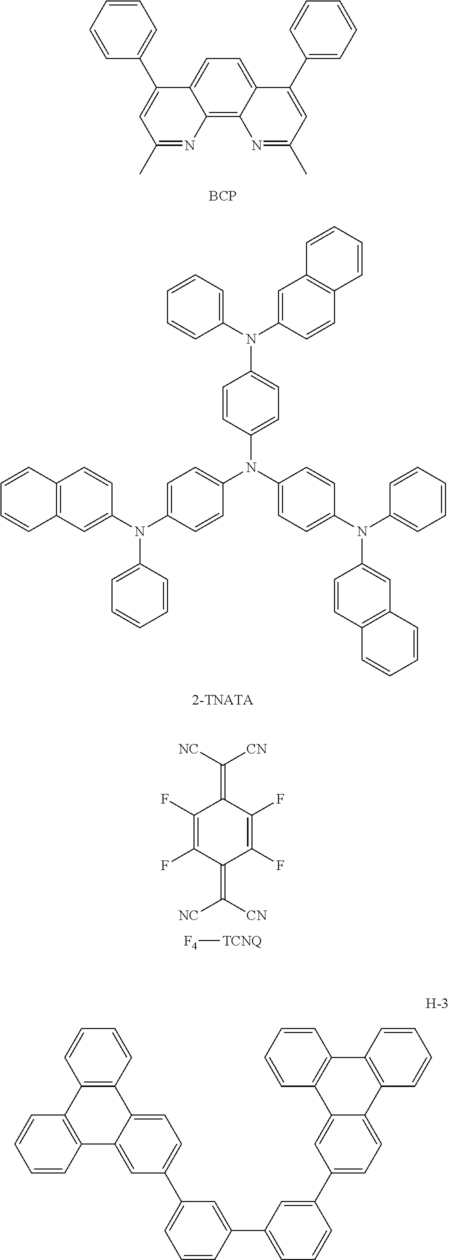

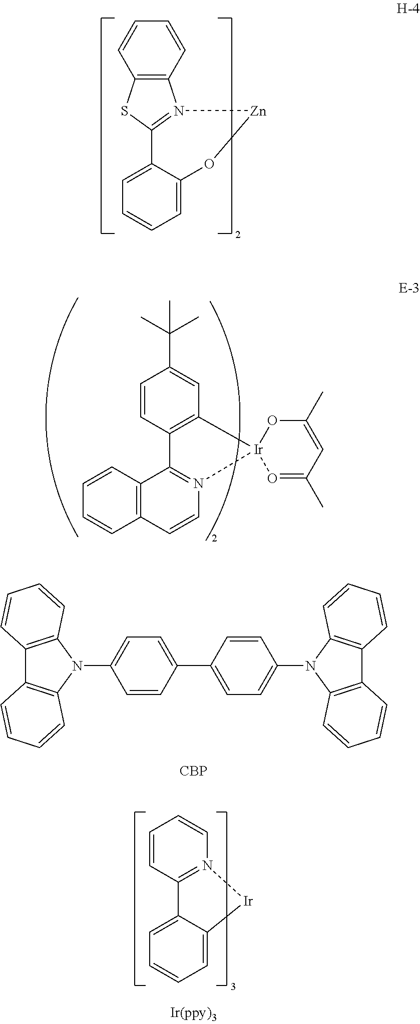

### INDUSTRIAL APPLICABILITY

The organic electroluminescence device of the present invention exhibits superior device properties after driving at high temperatures. Specifically, the organic electroluminescence device of the present invention exhibits superior external quantum efficiency and high durability during driving at high temperatures, and small variation in chromaticity and small increase in voltage after driving at high temperatures. In addition, the present invention provides an organic electroluminescence device with long lifespan, although a material having a carbazole group is used as a host material.

Although the present invention has been described in detail with reference to specific embodiments, it will be apparent to those skilled in the art that various variations and modifications are possible within the spirit and scope of the present invention.

This application claims the benefit of Japanese Patent Application No. 2010-007534, filed on Jan. 15, 2010, and Japanese Patent Application No. 2010-116664, filed on May 20, 2010, which are herein incorporated by reference as if fully set forth herein.

### EXPLANATION OF REFERENCE NUMERALS AND SYMBOLS

2 . . . Substrate
3 . . . Anode
4 . . . Hole injection layer
5 . . . Hole transporting layer
6 . . . Light emitting layer
7 . . . Hole block layer
8 . . . Electron transporting layer
9 . . . Cathode
10 . . . Organic electroluminescence device (organic EL device)
11 . . . Organic layer
12 . . . Protective layer
14 . . . Adhesive layer
16 . . . Sealing container
20 . . . Light emission apparatus
30 . . . Light scattering member
30A . . . Light incident surface
30B . . . Light emission surface
31 . . . Transparent substrate
32 . . . Particle
40 . . . Illumination apparatus

1. An organic electroluminescence device, comprising on a substrate:
a pair of electrodes; and
at least one layer of an organic layer including a light emitting layer disposed between the electrodes,
wherein the light emitting layer contains at least one compound represented by Formula (PQ-1), and
any layer of the at least one layer of an organic layer contains at least one compound represented by Formula (1): 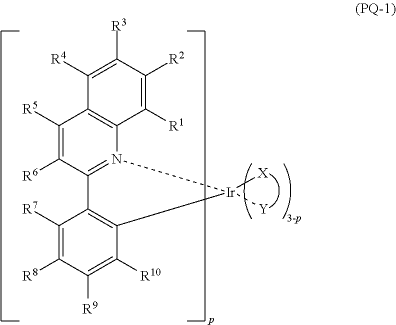
wherein in Formula (PQ-1), each of R¹ to R¹⁰ independently represents a hydrogen atom, an alkyl group, a cycloalkyl group, an aryl group, a cyano group or a fluorine atom, and substituents represented by R¹ to R¹⁰ may be combined together to form a ring, provided that all of the substituents represented by R¹ to R¹⁰ are not a hydrogen atom at the same time;
(X-Y) represents a monoanionic bidentate ligand; and
p represents an integer of 1 to 3: 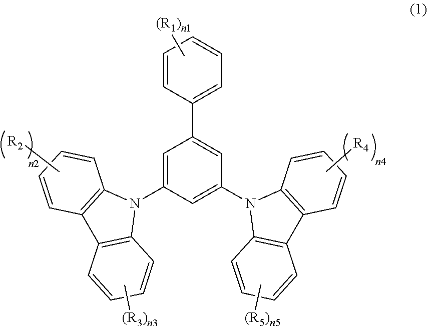
wherein in Formula (1), R₁ represents an alkyl group, an aryl group or a silyl group and may further have a substituent Z, provided that R₁ does not represent a carbazolyl group or a perfluoroalkyl alkyl group, and when R₁ is present in plural, each of a plurality of R₁'s may be the same as or different from every other R₁, and a plurality of R₁'s may be combined together to form an aryl ring which may have a substituent Z;
each of R₂ to R₅ independently represents an alkyl group, an aryl group, a silyl group, a cyano group or a fluorine atom and may further have a substituent Z, and when each of R₂ to R₅ is present in plural, each of a plurality of R₂'s to a plurality of R₅'s may be the same as or different from every other R₂ to R₅, respectively;
the substituent Z represents an alkyl group, an alkenyl group, an aryl group, an aromatic heterocyclic group, an alkoxy group, a phenoxy group, a fluorine atom, a silyl group, an amino group, a cyano group or a group formed by a combination thereof, and a plurality of the substituent Z's may be combined together to form an aryl group;
n1 represents an integer of 0 to 5; and
each of n2 to n5 independently represents an integer of 0 to 4.

2. The organic electroluminescence device according to claim 1,
wherein, in Formula (PQ-1), p is 2.

3. The organic electroluminescence device according to claim 1,
wherein in Formula (PQ-1), the monoanionic bidentate ligand (X-Y) is represented by the following Formula (PQL-1): 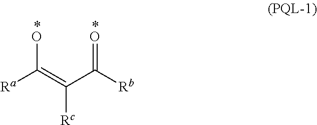
wherein in Formula (PQL-1), each of R^{a} to R^{c} independently represents a hydrogen atom or an alkyl group; and
* represents a position coordinated to iridium.

4. The organic electroluminescence device according to claim 1,
wherein in Formula (PQ-1), R¹ to R⁶ represent a hydrogen atom, each of R⁷ to R¹⁰ independently represents a hydrogen atom, an alkyl group or an aryl group, and at least one of R⁷ to R¹⁰ represents an alkyl group or an aryl group.

5. The organic electroluminescence device according to claim 1,
wherein the compound represented by Formula (1) is used in the light emitting layer.

6. The organic electroluminescence device according to claim 1,
wherein the compound represented by Formula (1) is represented by the following Formula (2): 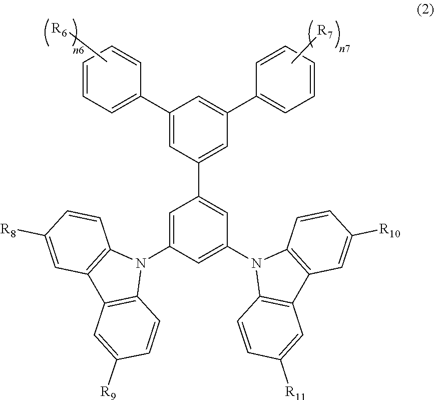
wherein, in Formula (2), each of R₆ and R₇ independently represents an alkyl group which may have a substituent Z, an aryl group which may have an alkyl group, a cyano group or a fluorine atom, and when each of R₆ and R₇ is present in plural, each of a plurality of R₆'s and a plurality of R₇'s may be the same as or different from every other R₆ and R₇, respectively, and each of the plurality of R₆'s and the plurality of R₇'s may be combined together to form an aryl ring that may have a substituent Z;
each of n6 and n7 independently represents an integer of 0 to 5;
each of R₈ to R₁₁ independently represents a hydrogen atom, an alkyl group which may have a substituent Z, an aryl group which may have an alkyl group, a silyl group which may have a substituent Z, a cyano group or a fluorine atom; and
the substituent Z represents an alkyl group, an alkenyl group, an aryl group, an aromatic heterocyclic group, an alkoxy group, a phenoxy group, a fluorine atom, a silyl group, an amino group, a cyano group or a group formed by a combination thereof, and a plurality of substituent Z's may be combined together to form an aryl group.

7. The organic electroluminescence device according to claim 6,
wherein in Formula (PQ-1), R¹to R⁶ represent a hydrogen atom, each of R⁷ to R¹⁰ independently represents a hydrogen atom, an alkyl group or an aryl group, at least one of R⁷ to R¹⁰ represents an alkyl group or an aryl group, p is 2 and the monoanionic bidentate ligand (X-Y) is represented by Formula (PQL-1): 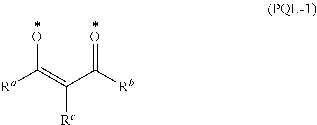
in Formula (PQL-1), each of R^{a} and R^{b} independently represents an alkyl group, R^{c} represents a hydrogen atom, and * represents a position coordinated to iridium, and in Formula (2), each of R₆ and R₇ independently represents an alkyl group or an aryl group which may have an alkyl group, each of n6 and n7 independently represents an integer of 0 to 2, each of R₈ to R₁₁ independently represents a hydrogen atom, an alkyl group, an aryl group which may have an alkyl group, a silyl group substituted by an alkyl group or a phenyl group, a cyano group or a fluorine atom.

8. The organic electroluminescence device according to claim 1, further comprising:
an electron injection layer disposed between the electrodes,
wherein the electron injection layer contains an electron donating dopant.

9. The organic electroluminescence device according to claim 1, further comprising:
a hole injection layer disposed between the electrodes,
wherein the hole injection layer contains a hole accepting dopant.

10. The organic electroluminescence device according to claim 1,
wherein at least one layer of the organic layer disposed between the pair of electrodes is formed by a solution coating process.

11. A light emitting layer, comprising:
a compound represented by Formula (PQ-1) and a compound represented by Formula (1): 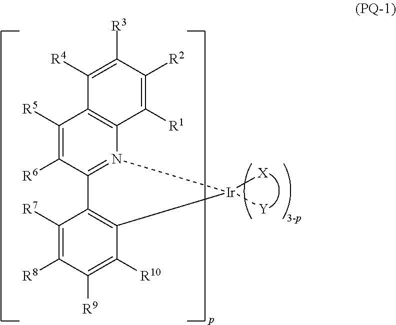
wherein in Formula (PQ-1), each of R¹ to R¹⁰ independently represents a hydrogen atom, an alkyl group, a cycloalkyl group, an aryl group, a cyano group or a fluorine atom, and substituents represented by R¹ to R¹⁰ may be combined together to form a ring, provided that all of the substituents represented by R¹ to R¹⁰ are not a hydrogen atom at the same time;
(X-Y) represents a monoanionic bidentate ligand; and
p represents an integer of 1 to 3: 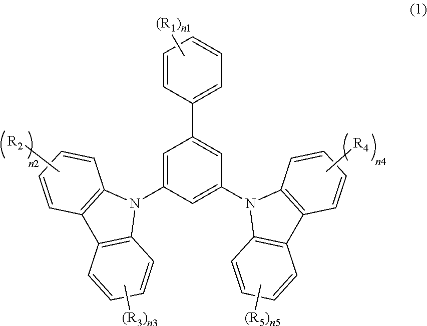
wherein in Formula (1), R₁ represents an alkyl group, an aryl group or a silyl group and may further have a substituent Z, provided that R₁ does not represent a carbazolyl group or a perfluoroalkyl alkyl group, and when R₁ is present in plural, each of a plurality of R₁'s may be the same as or different from every other R₁, and a plurality of R₁'s may be combined together to form an aryl ring which may have a substituent Z;
each of R₂ to R₅ independently represents an alkyl group, an aryl group, a silyl group, a cyano group or a fluorine atom and may further have a substituent Z, and when each of R₂ to R₅ is present in plural, each of a plurality of R₂'s to a plurality of R₅'s may be the same as or different from every other R₂ to R₅, respectively;
the substituent Z represents an alkyl group, an alkenyl group, an aryl group, an aromatic heterocyclic group, an alkoxy group, a phenoxy group, a fluorine atom, a silyl group, an amino group, a cyano group or a group formed by a combination thereof, and a plurality of the substituent Z's may be combined together to form an aryl group;
n1 represents an integer of 0 to 5; and
each of n2 to n5 independently represents an integer of 0 to 4.

12. A composition, comprising:
a compound represented by Formula (PQ-1) and a compound represented by Formula (1): 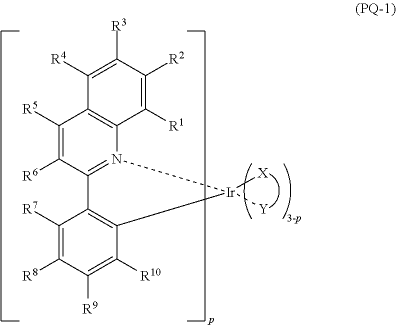
wherein in Formula (PQ-1), each of R¹ to R¹⁰ independently represents a hydrogen atom, an alkyl group, a cycloalkyl group, an aryl group, a cyano group or a fluorine atom, and substituents represented by R¹ to R¹⁰ may be combined together to form a ring, provided that all of the substituents represented by R¹ to R¹⁰ are not a hydrogen atom at the same time;
(X-Y) represents a monoanionic bidentate ligand; and
p represents an integer of 1 to 3: 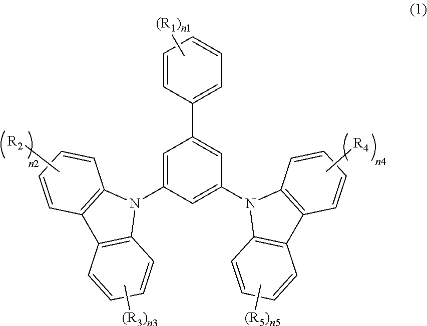
wherein in Formula (1), R₁ represents an alkyl group, an aryl group or a silyl group and may further have a substituent Z, provided that R₁ does not represent a carbazolyl group or a perfluoroalkyl alkyl group, and when R₁ is present in plural, each of a plurality of R₁'s may be the same as or different from every other R₁, and a plurality of R₁'s may be combined together to form an aryl ring which may have a substituent Z;
each of R₂ to R₅ independently represents an alkyl group, an aryl group, a silyl group, a cyano group or a fluorine atom and may further have a substituent Z, and when each of R₂ to R₅ is present in plural, each of a plurality of R₂'s to a plurality of R₅'s may be the same as or different from every other R₂ to R₅, respectively;
the substituent Z represents an alkyl group, an alkenyl group, an aryl group, an aromatic heterocyclic group, an alkoxy group, a phenoxy group, a fluorine atom, a silyl group, an amino group, a cyano group or a group formed by a combination thereof, and a plurality of the substituent Z's may be combined together to form an aryl group;
n1 represents an integer of 0 to 5; and
each of n2 to n5 independently represents an integer of 0 to 4.

13. A light emission apparatus using the organic electroluminescence device according to claim 1.

14. A display apparatus using the organic electroluminescence device according to claim 1.

15. An illumination apparatus using the organic electroluminescence device according to claim 1.
